SEQUENCE LISTING (122 sequences)

SEQ ID NO: 1 - DNA, 363 bp, synthetic construct
  misc_feature 1..363: note = Synthetic: Ab1,VH DNA
  source 1..363: mol_type = other DNA
gaggtccaacttgtggagagtggtggcggattagttcagccaggccgttcattgcgtttatcttgcgcggcgagcggatttacttttgacgattatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttagcacggcatctagcttggactattgggggcagggtacgttagtcaccgtttctagc

SEQ ID NO: 2 - protein, 121 aa, synthetic construct [sequence omitted]

SEQ ID NO: 3 - DNA, 321 bp, synthetic construct
  misc_feature 1..321: note = Synthetic: Ab1,VL DNA
  source 1..321: mol_type = other DNA
gatattcagatgactcaatccccttcgtctttaagcgcgagcgttggagatcgtgtgaccatcacctgccgtgcgtcccaaggcattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 4 - protein, 107 aa, synthetic construct [sequence omitted]

SEQ ID NO: 5 - DNA, 363 bp, synthetic construct
  misc_feature 1..363: note = Synthetic: A1,VH DNA
  source 1..363: mol_type = other DNA
gaggtccaacttgtggagagtggtggcggattagttcagccaggccgttcattgcgtttatcttgcgcggcgagcggatttacttttgaccagtatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttcctacggcatctagcttggactattgggggcagggtacgttagtcaccgtttctagc

SEQ ID NO: 6 - protein, 121 aa, synthetic construct
  REGION 1..121: note = Synthetic: A1,VH peptide
EVQLVESGGGLVQPGRSLRLSCAASGFTFDQYAMHWVRQAPGKGLEWVSAITWNSGHIDYADSVEGRFTISRDNAKNSLYLQMNSLRAEDTAVYYCAKVSYLPTASSLDYWGQGTLVTVSS

SEQ ID NO: 7 - DNA, 321 bp, synthetic construct
  misc_feature 1..321: note = Synthetic: A1,VL DNA
  source 1..321: mol_type = other DNA
gatattcagatgactcaatccccttcgtctttaagcgcgagcgttggagatcgtgtgaccatcacctgccgtgcgtcccaagagattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 8 - protein, 107 aa, synthetic construct
  REGION 1..107: note = Synthetic: A1,VL peptide
DIQMTQSPSSLSASVGDRVTITCRASQEIRNYLAWYQQKPGKAPKLLIYAASTLQSGVPSRFSGSGSGTDFTLTISSLQPEDVATYYCQRYNRAPYTFGQGTKVEIK

SEQ ID NO: 9 - DNA, 363 bp, synthetic construct
  misc_feature 1..363: note = Synthetic: cb1-3,VH DNA
  source 1..363: mol_type = other DNA
gaggtccaacttgtggagagtggtggcggattagttcagccaggccgttcattgcgtttatcttgcgcggcgagcggatttacttttgaccagtatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttagcacggcatctagcttggactattgggggcagggtacgttagtcaccgtttctagc

SEQ ID NO: 10 - protein, 121 aa, synthetic construct
  REGION 1..121: note = Synthetic: cb1-3,VH peptide
EVQLVESGGGLVQPGRSLRLSCAASGFTFDQYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 11 - DNA, 321 bp, synthetic construct
  misc_feature 1..321: note = Synthetic: cb1-3,VL DNA
  source 1..321: mol_type = other DNA
gatattcagatgactcaatccccttcgtctttaagcgcgagcgttggagatcgtgtgaccatcacctgccatgcgtcccgtaagattcgcaattacctggcttggtatcaacagaaacctggtaaggcccccaaattattgatctatgcggcttcgtctttgctgcgtggcgtgccctctcgcttcagcggcagcggatctggcaccgatttcaccttaactatcagctctttacagccagaagacgtagcgacgtattattgccagcgttatgacaagcccccgtacacgtttgggcaaggaacgaaggttgaaatcaag

SEQ ID NO: 12 - protein, 107 aa, synthetic construct
  REGION 1..107: note = Synthetic: cb1-3,VL peptide
DIQMTQSPSSLSASVGDRVTITCHASRKIRNYLAWYQQKPGKAPKLLIYAASSLLRGVPSRFSGSGSGTDFTLTISSLQPEDVATYYCQRYDKPPYTFGQGTKVEIK

SEQ ID NO: 13 - DNA, 363 bp, synthetic construct
  misc_feature 1..363: note = Synthetic: 4.2a-6,VH DNA
  source 1..363: mol_type = other DNA
gaggtccaacttgtggagagtggtggcggattagttcagccaggccgttcattgcgtttatcttgcgcggcgagcggatttacttttggccagtttgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttagcacggcatctagcttggactattgggggcagggtacgttagtcaccgtttctagc

SEQ ID NO: 14 - protein, 121 aa, synthetic construct
  REGION 1..121: note = Synthetic: 4.2a-6,VH peptide
EVQLVESGGGLVQPGRSLRLSCAASGFTFGQFAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 15 - DNA, 321 bp, synthetic construct
  misc_feature 1..321: note = Synthetic: 4.2a-6,VL DNA
  source 1..321: mol_type = other DNA
gatattcagatgactcaatccccttcgtctttaagcgcgagcgttggagatcgtgtgaccatcacctgccgtgcgtcccaacgcctgcgcaattacctggcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccagcgttatattcagccgccgtacacgtttgggcaaggaacgaaggttgaaatcaag

SEQ ID NO: 16 - protein, 107 aa, synthetic construct
  REGION 1..107: note = Synthetic: 4.2a-6,VL peptide
DIQMTQSPSSLSASVGDRVTITCRASQRLRNYLAWYQQKPGKAPKLLIGAASSTISGVPSRFSGSGSGTDFTLTISSLQPEDVATYYCQRYIQPPYTFGQGTKVEIK

SEQ ID NO: 17 - DNA, 354 bp, synthetic construct
  misc_feature 1..354: note = Synthetic: Ab4,VH DNA
  source 1..354: mol_type = other DNA
gaagttcagcttgtggaaagcggaggcggtttagtccagccgggtggatcattacgtctttcttgcgccgcaagtggatatgtatttacagattacggcatgaattgggtacgccaagcgcctgggaaaggacttgagtggatgggctggatcaatacctatattggcgaacctatttatgccgattcagttaaaggacgttttacgttttctctggacacttctaagtccactgcttacttgcaaatgaacagcttacgtgctgaggacaccgccgtgtattactgtgcacgcggctatcgctcctacgctatggactactggggtcagggcacgttggtcacagttagtagc

SEQ ID NO: 18 - protein, 118 aa, synthetic construct
  REGION 1..118: note = Synthetic: Ab4,VH peptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 19 - DNA, 321 bp, synthetic construct
  misc_feature 1..321: note = Synthetic: Ab4,VL DNA
  source 1..321: mol_type = other DNA
gatattcaaatgacacagtccccttcgagcttgtccgcttcagttggtgaccgtgtcactatcacatgtaaagcctcacagaatgtaggaacgaatgtagcttggtatcaacagaaacccggcaaggcaccgaaggcgctgatttattccgcctctttcctgtactctggcgtcccctaccgttttagtggatctggatccgggacggatttcactttaactatctcctcgcttcagcctgaagattttgcgacctactattgtcagcagtataatatctacccccttacgttcggtcagggcaccaaggtagagatcaaa

SEQ ID NO: 20 - protein, 107 aa, synthetic construct
  REGION 1..107: note = Synthetic: Ab4,VL peptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 21 - DNA, 363 bp, synthetic construct
  misc_feature 1..363: note = Synthetic: Ab1 VH1,VH DNA
  source 1..363: mol_type = other DNA
gaggtccaacttgtggagagtggtggcggattagttcagccaggccgttcattgcgtttatcttgcgcggcgagcggatttacttttgacgattatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcatagcacggcatctagcttggactattgggggcagggtacgttagtcaccgtttctagc

SEQ ID NO: 22 - protein, 121 aa, synthetic construct
  REGION 1..121: note = Synthetic: Ab1 VH1,VH peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYHSTASSLDYWGQGTLVTVSS

SEQ ID NO: 23 - DNA, 363 bp, synthetic construct
  misc_feature 1..363: note = Synthetic: Ab1 VH2,VH DNA
  source 1..363: mol_type = other DNA
gaggtccaacttgtggagagtggtggcggattagttcagccaggccgttcattgcgtttatcttgcgcggcgagcggatttacttttgacgattatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttagcacggcacatcatttggactattgggggcagggtacgttagtcaccgtttctagc

SEQ ID NO: 24 - protein, 121 aa, synthetic construct
  REGION 1..121: note = Synthetic: Ab1 VH2,VH peptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTAHHLDYWGQGTLVTVSS

SEQ ID NO: 25 - DNA, 321 bp, synthetic construct
  misc_feature 1..321: note = Synthetic: Ab1 VL2,VL DNA
  source 1..321: mol_type = other DNA
gatattcagatgactcaatccccttcgtctttaagcgcgagcgttggagatcgtgtgaccatcacctgccgtgcgtcccaaggcattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcattatcatcgcgcaccgtacacgtttgggcaaggaacgaaggttgaaatcaag

SEQ ID NO: 26 - protein, 107 aa, synthetic construct
  REGION 1..107: note = Synthetic: Ab1 VL2,VL peptide
DIQMTQSPSSLSASVGDRVTITCRASQGIRNYLAWYQQKPGKAPKLLIYAASTLQSGVPSRFSGSGSGTDFTLTISSLQPEDVATYYCQHYHRAPYTFGQGTKVEIK

SEQ ID NO: 27 - DNA, 321 bp, synthetic construct
  misc_feature 1..321: note = Synthetic: Ab1 VL4,VL DNA
  source 1..321: mol_type = other DNA
gatattcagatgactcaatccccttcgtctttaagcgcgagcgttggagatcgtgtgaccatcacctgccgtgcgtcccaaggcattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccatcattatcatcgcgcaccgtacacgtttgggcaaggaacgaaggttgaaatcaag

SEQ ID NO: 28 - protein, 107 aa, synthetic construct
  REGION 1..107: note = Synthetic: Ab1 VL4,VL peptide
DIQMTQSPSSLSASVGDRVTITCRASQGIRNYLAWYQQKPGKAPKLLIYAASTLQSGVPSRFSGSGSGTDFTLTISSLQPEDVATYYCHHYHRAPYTFGQGTKVEIK

SEQ ID NO: 29 - DNA, 363 bp, synthetic construct
  misc_feature 1..363: note = Synthetic: 4.2a-6 VH1,VH DNA
  source 1..363: mol_type = other DNA
gaggtccaacttgtggagagtggtggcggattagttcagccaggccgttcattgcgtttatcttgcgcggcgagcggatttacttttggccagtttgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcatagcacggcatctagcttggactattgggggcagggtacgttagtcaccgtttctagc

SEQ ID NO: 30 - protein, 121 aa, synthetic construct
  REGION 1..121: note = Synthetic: 4.2a-6 VH1,VH peptide
EVQLVESGGGLVQPGRSLRLSCAASGFTFGQFAMHWVRQAPGKGLEWVSAITWNSGHIDYADSVEGRFTISRDNAKNSLYLQMNSLRAEDTAVYYCAKVSYHSTASSLDYWGQGTLVTVSS

SEQ ID NO: 31 - DNA, 363 bp, synthetic construct
  misc_feature 1..363: note = Synthetic: 4.2a-6 VH2,VH DNA
  source 1..363: mol_type = other DNA
gaggtccaacttgtggagagtggtggcggattagttcagccaggccgttcattgcgtttatcttgcgcggcgagcggatttacttttggccagtttgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttagcacggcacatcatttggactattgggggcagggtacgttagtcaccgtttctagc

SEQ ID NO: 32 - protein, 121 aa, synthetic construct
  REGION 1..121: note = Synthetic: 4.2a-6 VH2,VH peptide
EVQLVESGGGLVQPGRSLRLSCAASGFTFGQFAMHWVRQAPGKGLEWVSAITWNSGHIDYADSVEGRFTISRDNAKNSLYLQMNSLRAEDTAVYYCAKVSYLSTAHHLDYWGQGTLVTVSS

SEQ ID NO: 33 - DNA, 363 bp, synthetic construct
  misc_feature 1..363: note = Synthetic: 4.2a-6 VH3,VH DNA
  source 1..363: mol_type = other DNA
gaggtccaacttgtggagagtggtggcggattagttcagccaggccgttcattgcgtttatcttgcgcggcgagcggatttacttttggccagtttgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgcattatcatagcacggcatctagcttggactattgggggcagggtacgttagtcaccgtttctagc

SEQ ID NO: 34 - protein, 121 aa, synthetic construct
  REGION 1..121: note = Synthetic: 4.2a-6 VH3,VH peptide
EVQLVESGGGLVQPGRSLRLSCAASGFTFGQFAMHWVRQAPGKGLEWVSAITWNSGHIDYADSVEGRFTISRDNAKNSLYLQMNSLRAEDTAVYYCAKVHYHSTASSLDYWGQGTLVTVSS

SEQ ID NO: 35 - DNA, 363 bp, synthetic construct
  misc_feature 1..363: note = Synthetic: 4.2a-6-H,VH DNA
  source 1..363: mol_type = other DNA
gaggtccaacttgtggagagtggtggcggattagttcagccaggccgttcattgcgtttatcttgcgcggcgagcggatttacttttggccagtttgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttagcacggcatctagccatgactattgggggcagggtacgttagtcaccgtttctagc

SEQ ID NO: 36 - protein, 121 aa, synthetic construct
  REGION 1..121: note = Synthetic: 4.2a-6-H,VH peptide
EVQLVESGGGLVQPGRSLRLSCAASGFTFGQFAMHWVRQAPGKGLEWVSAITWNSGHIDYADSVEGRFTISRDNAKNSLYLQMNSLRAEDTAVYYCAKVSYLSTASSHDYWGQGTLVTVSS

SEQ ID NO: 37 - DNA, 321 bp, synthetic construct
  misc_feature 1..321: note = Synthetic: 4.2a-6-H,VL DNA
  source 1..321: mol_type = other DNA
gatattcagatgactcaatccccttcgtctttaagcgcgagcgttggagatcgtgtgaccatcacctgccgtgcgtcccaacgcctgcgcaattacctggcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccatcgtcatattcagccgccgtacacgtttgggcaaggaacgaaggttgaaatcaag

SEQ ID NO: 38 - protein, 107 aa, synthetic construct
  REGION 1..107: note = Synthetic: 4.2a-6-H,VL peptide
DIQMTQSPSSLSASVGDRVTITCRASQRLRNYLAWYQQKPGKAPKLLIGAASSTISGVPSRFSGSGSGTDFTLTISSLQPEDVATYYCHRHIQPPYTFGQGTKVEIK

SEQ ID NO: 39 - DNA, 321 bp, synthetic construct
  misc_feature 1..321: note = Synthetic: 4.2a-6 VL1,VL DNA
  source 1..321: mol_type = other DNA
gatattcagatgactcaatccccttcgtctttaagcgcgagcgttggagatcgtgtgaccatcacctgccgtgcgtcccaacgcctgcgcaatcatctggcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccagcgttatattcagccgccgtacacgtttgggcaaggaacgaaggttgaaatcaag

SEQ ID NO: 40 - protein, 107 aa, synthetic construct
  REGION 1..107: note = Synthetic: 4.2a-6 VL1,VL peptide
DIQMTQSPSSLSASVGDRVTITCRASQRLRNHLAWYQQKPGKAPKLLIGAASSTISGVPSRFSGSGSGTDFTLTISSLQPEDVATYYCQRYIQPPYTFGQGTKVEIK

SEQ ID NO: 41 - DNA, 321 bp, synthetic construct
  misc_feature 1..321: note = Synthetic: 4.2a-6 VL2,VL DNA
  source 1..321: mol_type = other DNA
gatattcagatgactcaatccccttcgtctttaagcgcgagcgttggagatcgtgtgaccatcacctgccgtgcgtcccaacgcctgcgcaattacctggcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccagcattatcatcagccgccgtacacgtttgggcaaggaacgaaggttgaaatcaag

SEQ ID NO: 42 - protein, 107 aa, synthetic construct
  REGION 1..107: note = Synthetic: 4.2a-6 VL2,VL peptide
DIQMTQSPSSLSASVGDRVTITCRASQRLRNYLAWYQQKPGKAPKLLIGAASSTISGVPSRFSGSGSGTDFTLTISSLQPEDVATYYCQHYHQPPYTFGQGTKVEIK

SEQ ID NO: 43 - DNA, 321 bp, synthetic construct
  misc_feature 1..321: note = Synthetic: 4.2a-6 VL4,VL DNA
  source 1..321: mol_type = other DNA
gatattcagatgactcaatccccttcgtctttaagcgcgagcgttggagatcgtgtgaccatcacctgccgtgcgtcccaacgcctgcgcaattacctggcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccatcattatcatcagccgccgtacacgtttgggcaaggaacgaaggttgaaatcaag

SEQ ID NO: 44 - protein, 107 aa, synthetic construct
  REGION 1..107: note = Synthetic: 4.2a-6 VL4,VL peptide
DIQMTQSPSSLSASVGDRVTITCRASQRLRNYLAWYQQKPGKAPKLLIGAASSTISGVPSRFSGSGSGTDFTLTISSLQPEDVATYYCHHYHQPPYTFGQGTKVEIK

SEQ ID NO: 45 - DNA, 321 bp, synthetic construct
  misc_feature 1..321: note = Synthetic: 4.2a-6 VL5,VL DNA
  source 1..321: mol_type = other DNA
gatattcagatgactcaatccccttcgtctttaagcgcgagcgttggagatcgtgtgaccatcacctgccgtgcgtcccaacgcctgcgcaatcatcatgcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccagcgttatattcagccgccgtacacgtttgggcaaggaacgaaggttgaaatcaag

SEQ ID NO: 46 - protein, 107 aa, synthetic construct
  REGION 1..107: note = Synthetic: 4.2a-6 VL5,VL peptide
DIQMTQSPSSLSASVGDRVTITCRASQRLRNHHAWYQQKPGKAPKLLIGAASSTISGVPSRFSGSGSGTDFTLTISSLQPEDVATYYCQRYIQPPYTFGQGTKVEIK

SEQ ID NO: 47 - DNA, 321 bp, synthetic construct
  misc_feature 1..321: note = Synthetic: 4.2a-6 VL7,VL DNA
  source 1..321: mol_type = other DNA
gatattcagatgactcaatccccttcgtctttaagcgcgagcgttggagatcgtgtgaccatcacctgccgtgcgtcccaacgcctgcgcaatcacctggcttggtatcaacagaaacctggtaaggcccccaaattattgatcggcgcggcttcgagcaccattagcggcgtgccctctcgcttcagcggcagcggatctggcaccgatttcaccttaactatcagctctttacagccagaagacgtagcgacgtattattgccagcgttatattcacccgccgtacacgtttgggcaaggaacgaaggttgaaatcaag

SEQ ID NO: 48 - protein, 107 aa, synthetic construct
  REGION 1..107: note = Synthetic: 4.2a-6 VL7,VL peptide
DIQMTQSPSSLSASVGDRVTITCRASQRLRNHLAWYQQKPGKAPKLLIGAASSTISGVPSRFSGSGSGTDFTLTISSLQPEDVATYYCQRYIHPPYTFGQGTKVEIK

SEQ ID NO: 49 - DNA, 363 bp, synthetic construct
  misc_feature 1..363: note = Synthetic: A1 VH1,VH DNA
  source 1..363: mol_type = other DNA
gaggtccaacttgtggagagtggtggcggattagttcagccaggccgttcattgcgtttatcttgcgcggcgagcggatttacttttgaccagtatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcatcctacggcatctagcttggactattgggggcagggtacgttagtcaccgtttctagc

SEQ ID NO: 50 - protein, 121 aa, synthetic construct
  REGION 1..121: note = Synthetic: A1 VH1,VH peptide
EVQLVESGGGLVQPGRSLRLSCAASGFTFDQYAMHWVRQAPGKGLEWVSAITWNSGHIDYADSVEGRFTISRDNAKNSLYLQMNSLRAEDTAVYYCAKVSYHPTASSLDYWGQGTLVTVSS

SEQ ID NO: 51 - DNA, 363 bp, synthetic construct
  misc_feature 1..363: note = Synthetic: A1 VH2,VH DNA
  source 1..363: mol_type = other DNA
gaggtccaacttgtggagagtggtggcggattagttcagccaggccgttcattgcgtttatcttgcgcggcgagcggatttacttttgaccagtatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgtcttatcttcctacggcacatcatttggactattgggggcagggtacgttagtcaccgtttctagc

SEQ ID NO: 52 - protein, 121 aa, synthetic construct
  REGION 1..121: note = Synthetic: A1 VH2,VH peptide
EVQLVESGGGLVQPGRSLRLSCAASGFTFDQYAMHWVRQAPGKGLEWVSAITWNSGHIDYADSVEGRFTISRDNAKNSLYLQMNSLRAEDTAVYYCAKVSYLPTAHHLDYWGQGTLVTVSS

SEQ ID NO: 53 - DNA, 363 bp, synthetic construct
  misc_feature 1..363: note = Synthetic: A1 VH3,VH DNA
  source 1..363: mol_type = other DNA
gaggtccaacttgtggagagtggtggcggattagttcagccaggccgttcattgcgtttatcttgcgcggcgagcggatttacttttgaccagtatgctatgcactgggtacgccaagcacctggaaagggtctggagtgggtgagtgcaatcacatggaatagcggtcacattgactacgcagactccgttgaaggtcgctttaccattagccgtgacaacgcgaagaattctctgtatttacagatgaattccttacgcgcggaggatactgccgtctattattgtgctaaagtgcattatcatcctacggcatctagcttggactattgggggcagggtacgttagtcaccgtttctagc

SEQ ID NO: 54 - protein, 121 aa, synthetic construct
  REGION 1..121: note = Synthetic: A1 VH3,VH peptide
EVQLVESGGGLVQPGRSLRLSCAASGFTFDQYAMHWVRQAPGKGLEWVSAITWNSGHIDYADSVEGRFTISRDNAKNSLYLQMNSLRAEDTAVYYCAKVHYHPTASSLDYWGQGTLVTVSS

SEQ ID NO: 55 - DNA, 321 bp, synthetic construct
  misc_feature 1..321: note = Synthetic: A1 VL1,VL DNA
  source 1..321: mol_type = other DNA
gatattcagatgactcaatccccttcgtctttaagcgcgagcgttggagatcgtgtgaccatcacctgccgtgcgtcccaagagattcgcaatcatctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 56 - protein, 107 aa, synthetic construct
  REGION 1..107: note = Synthetic: A1 VL1,VL peptide
DIQMTQSPSSLSASVGDRVTITCRASQEIRNHLAWYQQKPGKAPKLLIYAASTLQSGVPSRFSGSGSGTDFTLTISSLQPEDVATYYCQRYNRAPYTFGQGTKVEIK

SEQ ID NO: 57 - DNA, 321 bp, synthetic construct
  misc_feature 1..321: note = Synthetic: A1 VL2,VL DNA
  source 1..321: mol_type = other DNA
gatattcagatgactcaatccccttcgtctttaagcgcgagcgttggagatcgtgtgaccatcacctgccgtgcgtcccaagagattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcattatcatcgcgcaccgtacacgtttgggcaaggaacgaaggttgaaatcaag

SEQ ID NO: 58 - protein, 107 aa, synthetic construct
  REGION 1..107: note = Synthetic: A1 VL2,VL peptide
DIQMTQSPSSLSASVGDRVTITCRASQEIRNYLAWYQQKPGKAPKLLIYAASTLQSGVPSRFSGSGSGTDFTLTISSLQPEDVATYYCQHYHRAPYTFGQGTKVEIK

SEQ ID NO: 59 - DNA, 321 bp, synthetic construct
  misc_feature 1..321: note = Synthetic: A1 VL3,VL DNA
  source 1..321: mol_type = other DNA
gatattcagatgactcaatccccttcgtctttaagcgcgagcgttggagatcgtgtgaccatcacctgccgtgcgtcccaagagattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcattatcatcgcgcaccgtaccattttgggcaaggaacgaaggttgaaatcaag

SEQ ID NO: 60 - protein, 107 aa, synthetic construct
  REGION 1..107: note = Synthetic: A1 VL3,VL peptide
DIQMTQSPSSLSASVGDRVTITCRASQEIRNYLAWYQQKPGKAPKLLIYAASTLQSGVPSRFSGSGSGTDFTLTISSLQPEDVATYYCQHYHRAPYHFGQGTKVEIK

SEQ ID NO: 61 - DNA, 321 bp, synthetic construct
  misc_feature 1..321: note = Synthetic: A1 VL4,VL DNA
  source 1..321: mol_type = other DNA
gatattcagatgactcaatccccttcgtctttaagcgcgagcgttggagatcgtgtgaccatcacctgccgtgcgtcccaagagattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccatcattatcatcgcgcaccgtacacgtttgggcaaggaacgaaggttgaaatcaag

SEQ ID NO: 62 - protein, 107 aa, synthetic construct
  REGION 1..107: note = Synthetic: A1 VL4,VL peptide
DIQMTQSPSSLSASVGDRVTITCRASQEIRNYLAWYQQKPGKAPKLLIYAASTLQSGVPSRFSGSGSGTDFTLTISSLQPEDVATYYCHHYHRAPYTFGQGTKVEIK

SEQ ID NO: 63 - DNA, 321 bp, synthetic construct
  misc_feature 1..321: note = Synthetic: A1 VL5,VL DNA
  source 1..321: mol_type = other DNA
gatattcagatgactcaatccccttcgtctttaagcgcgagcgttggagatcgtgtgaccatcacctgccgtgcgtcccaagagattcgcaatcatcatgcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 64 - protein, 107 aa, synthetic construct
  REGION 1..107: note = Synthetic: A1 VL5,VL peptide
DIQMTQSPSSLSASVGDRVTITCRASQEIRNHHAWYQQKPGKAPKLLIYAASTLQSGVPSRFSGSGSGTDFTLTISSLQPEDVATYYCQRYNRAPYTFGQGTKVEIK

SEQ ID NO: 65 - DNA, 321 bp, synthetic construct
  misc_feature 1..321: note = Synthetic: A1 VL6,VL DNA
  source 1..321: mol_type = other DNA
gatattcagatgactcaatccccttcgtctttaagcgcgagcgttggagatcgtgtgaccatcacctgccgtgcgtcccaagagattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgtcataaccgcgcaccgtacacgtttgggcaaggaacgaaggttgaaatcaag

SEQ ID NO: 66 - protein, 107 aa, synthetic construct
  REGION 1..107: note = Synthetic: A1 VL6,VL peptide
DIQMTQSPSSLSASVGDRVTITCRASQEIRNYLAWYQQKPGKAPKLLIYAASTLQSGVPSRFSGSGSGTDFTLTISSLQPEDVATYYCQRHNRAPYTFGQGTKVEIK

SEQ ID NO: 67 - DNA, 321 bp, synthetic construct
  misc_feature 1..321: note = Synthetic: A1 VL7,VL DNA
  source 1..321: mol_type = other DNA
gatattcagatgactcaatccccttcgtctttaagcgcgagcgttggagatcgtgtgaccatcacctgccgtgcgtcccaagagattcgcaatcatctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccatgcaccgtacacgtttgggcaaggaacgaaggttgaaatcaag

SEQ ID NO: 68 - protein, 107 aa, synthetic construct
  REGION 1..107: note = Synthetic: A1 VL7,VL peptide
DIQMTQSPSSLSASVGDRVTITCRASQEIRNHLAWYQQKPGKAPKLLIYAASTLQSGVPSRFSGSGSGTDFTLTISSLQPEDVATYYCQRYNHAPYTFGQGTKVEIK

SEQ ID NO: 69 - DNA, 321 bp, synthetic construct
  misc_feature 1..321: note = Synthetic: A1 LC Q27H,VL DNA
  source 1..321: mol_type = other DNA
gatattcagatgactcaatccccttcgtctttaagcgcgagcgttggagatcgtgtgaccatcacctgccgtgcgtcccatgagattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 70 - protein, 107 aa, synthetic construct
  REGION 1..107: note = Synthetic: A1 LC Q27H,VL peptide
DIQMTQSPSSLSASVGDRVTITCRASHEIRNYLAWYQQKPGKAPKLLIYAASTLQSGVPSRFSGSGSGTDFTLTISSLQPEDVATYYCQRYNRAPYTFGQGTKVEIK

SEQ ID NO: 71 - DNA, 321 bp, synthetic construct
  misc_feature 1..321: note = Synthetic: A1 LC G28H,VL DNA
  source 1..321: mol_type = other DNA
gatattcagatgactcaatccccttcgtctttaagcgcgagcgttggagatcgtgtgaccatcacctgccgtgcgtcccaacatattcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 72 - protein, 107 aa, synthetic construct
  REGION 1..107: note = Synthetic: A1 LC G28H,VL peptide
DIQMTQSPSSLSASVGDRVTITCRASQHIRNYLAWYQQKPGKAPKLLIYAASTLQSGVPSRFSGSGSGTDFTLTISSLQPEDVATYYCQRYNRAPYTFGQGTKVEIK

SEQ ID NO: 73 - DNA, 321 bp, synthetic construct
  misc_feature 1..321: note = Synthetic: A1 LC I29H,VL DNA
  source 1..321: mol_type = other DNA
gatattcagatgactcaatccccttcgtctttaagcgcgagcgttggagatcgtgtgaccatcacctgccgtgcgtcccaagagcatcgcaattacctggcttggtatcaacagaaacctggtaaggcccccaaattattgatctatgcggcttcgactttgcaaagcggcgtgccctctcgcttcagcggcagcggatctggcaccgatttcaccttaactatcagctctttacagccagaagacgtagcgacgtattattgccagcgttataaccgcgcaccgtacacgtttgggcaaggaacgaaggttgaaatcaag

SEQ ID NO: 74 - protein, 107 aa, synthetic construct
  REGION 1..107: note = Synthetic: A1 LC I29H,VL peptide
DIQMTQSPSSLSASVGDRVTITCRASQEHRNYLAWYQQKPGKAPKLLIYAASTLQSGVPSRFSGSGSGTDFTLTISSLQPEDVATYYCQRYNRAPYTFGQGTKVEIK

SEQ ID NO: 75 - protein, 5 aa, synthetic construct
  REGION 1..5: note = Synthetic
DYAMH

SEQ ID NO: 76 - protein, 17 aa, synthetic construct
  REGION 1..17: note = Synthetic
AITWNSGHIDYADSVEG

SEQ ID NO: 77 - protein, 12 aa, synthetic construct
  REGION 1..12: note = Synthetic
VSYLSTASSLDY

SEQ ID NO: 78 - protein, 11 aa, synthetic construct
  REGION 1..11: note = Synthetic
RASQGIRNYLA

SEQ ID NO: 79 - protein, 7 aa, synthetic construct
  REGION 1..7: note = Synthetic
AASTLQS

SEQ ID NO: 80 - protein, 9 aa, synthetic construct
  REGION 1..9: note = Synthetic
QRYNRAPYT

SEQ ID NO: 81 - protein, 5 aa, synthetic construct
  REGION 1..5: note = Synthetic
QYAMH

SEQ ID NO: 82 - protein, 12 aa, synthetic construct
  REGION 1..12: note = Synthetic
VSYLPTASSLDY

SEQ ID NO: 83 - protein, 11 aa, synthetic construct
  REGION 1..11: note = Synthetic
RASQEIRNYLA

SEQ ID NO: 84 - protein, 11 aa, synthetic construct
  REGION 1..11: note = Synthetic
HASRKIRNYLA

SEQ ID NO: 85 - protein, 7 aa, synthetic construct
  REGION 1..7: note = Synthetic
AASSLLR

SEQ ID NO: 86 - protein, 9 aa, synthetic construct
  REGION 1..9: note = Synthetic
QRYDKPPYT

SEQ ID NO: 87 - protein, 5 aa, synthetic construct
  REGION 1..5: note = Synthetic
QFAMH

SEQ ID NO: 88 - protein, 11 aa, synthetic construct
  REGION 1..11: note = Synthetic
RASQRLRNYLA

SEQ ID NO: 89 - protein, 7 aa, synthetic construct
  REGION 1..7: note = Synthetic
AASSTIS

SEQ ID NO: 90 - protein, 9 aa, synthetic construct
  REGION 1..9: note = Synthetic
QRYIQPPYT

SEQ ID NO: 91 - protein, 5 aa, synthetic construct
  REGION 1..5: note = Synthetic
DYGMN

SEQ ID NO: 92 - protein, 17 aa, synthetic construct
  REGION 1..17: note = Synthetic
WINTYIGEPIYADSVKG

SEQ ID NO: 93 - protein, 9 aa, synthetic construct
  REGION 1..9: note = Synthetic
GYRSYAMDY

SEQ ID NO: 94 - protein, 11 aa, synthetic construct
  REGION 1..11: note = Synthetic
KASQNVGTNVA

SEQ ID NO: 95 - protein, 7 aa, synthetic construct
  REGION 1..7: note = Synthetic
SASFLYS

SEQ ID NO: 96 - protein, 9 aa, synthetic construct
  REGION 1..9: note = Synthetic
QQYNIYPLT

SEQ ID NO: 97 - protein, 12 aa, synthetic construct
  REGION 1..12: note = Synthetic
VSYHSTASSLDY

SEQ ID NO: 98 - protein, 12 aa, synthetic construct
  REGION 1..12: note = Synthetic
VSYLSTAHHLDY

SEQ ID NO: 99 - protein, 9 aa, synthetic construct
  REGION 1..9: note = Synthetic
QHYHRAPYT

SEQ ID NO: 100 - protein, 9 aa, synthetic construct
  REGION 1..9: note = Synthetic
HHYHRAPYT

SEQ ID NO: 101 - protein, 12 aa, synthetic construct
  REGION 1..12: note = Synthetic
VHYHSTASSLDY

SEQ ID NO: 102 - protein, 12 aa, synthetic construct
  REGION 1..12: note = Synthetic
VSYLSTASSHDY

SEQ ID NO: 103 - protein, 9 aa, synthetic construct
  REGION 1..9: note = Synthetic
HRHIQPPYT

SEQ ID NO: 104 - protein, 11 aa, synthetic construct
  REGION 1..11: note = Synthetic
RASQRLRNHLA

SEQ ID NO: 105 - protein, 9 aa, synthetic construct
  REGION 1..9: note = Synthetic
QHYHQPPYT

SEQ ID NO: 106 - protein, 9 aa, synthetic construct
  REGION 1..9: note = Synthetic
HHYHQPPYT

SEQ ID NO: 107 - protein, 11 aa, synthetic construct
  REGION 1..11: note = Synthetic
RASQRLRNHHA

SEQ ID NO: 108 - protein, 9 aa, synthetic construct
  REGION 1..9: note = Synthetic
QRYIHPPYT

SEQ ID NO: 109 - protein, 12 aa, synthetic construct
  REGION 1..12: note = Synthetic
VSYHPTASSLDY

SEQ ID NO: 110 - protein, 12 aa, synthetic construct
  REGION 1..12: note = Synthetic
VSYLPTAHHLDY

SEQ ID NO: 111 - protein, 12 aa, synthetic construct
  REGION 1..12: note = Synthetic
VHYHPTASSLDY

SEQ ID NO: 112 - protein, 11 aa, synthetic construct
  REGION 1..11: note = Synthetic
RASQEIRNHLA

SEQ ID NO: 113 - protein, 9 aa, synthetic construct
  REGION 1..9: note = Synthetic
QHYHRAPYH

SEQ ID NO: 114 - protein, 11 aa, synthetic construct
  REGION 1..11: note = Synthetic
RASQEIRNHHA

SEQ ID NO: 115 - protein, 9 aa, synthetic construct
  REGION 1..9: note = Synthetic
QRHNRAPYT

SEQ ID NO: 116 - protein, 9 aa, synthetic construct
  REGION 1..9: note = Synthetic
QRYNHAPYT

SEQ ID NO: 117 - protein, 11 aa, synthetic construct
  REGION 1..11: note = Synthetic
RASHEIRNYLA

SEQ ID NO: 118 - protein, 11 aa, synthetic construct
  REGION 1..11: note = Synthetic
RASQHIRNYLA

SEQ ID NO: 119 - protein, 11 aa, synthetic construct
  REGION 1..11: note = Synthetic
RASQEHRNYLA

SEQ ID NO: 120 - protein, 451 aa, synthetic construct
  REGION 1..451: note = Synthetic: Ab1 HC
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 214 aa, synthetic construct
  REGION 1..214: note = Synthetic: Ab1 LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 233 aa, Homo sapiens
  REGION 1..233: note = misc_feature - Human TNF-ALPHA polypeptide sequence
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

(19) United States
(12) Patent Application Publication
(10) Pub. No.: US 2025/0388665 A1
(43) Pub. Date: Dec. 25, 2025

(54) ANTI-TNF-ALPHA ANTIBODIES AND COMPOSITIONS

(71) Applicant: ADAFRE BIOSCIENCES, LLC, Encinitas, CA (US)

(72) Inventors: Jeffry D. Watkins, Encinitas, CA (US); J. Monty Watkins, La Jolla, CA (US)

(73) Assignee: ADAFRE BIOSCIENCES, LLC, Encinitas, CA (US)

(21) Appl. No.: 18/878,712

(22) PCT Filed: Jun. 26, 2023
(86) PCT No.: PCT/US2023/069042

§ 371 Date: Dec. 24, 2024

Related U.S. Application Data
(60) Provisional application No. 63/356,138, filed on Jun. 28, 2022.

(51) Int. Cl. C07K 16/24; A61K 31/519; A61K 47/68; C07K 16/22

(52) U.S. Cl. CPC C07K 16/241; A61K 31/519; A61K 47/6803; A61K 47/6845; C07K 16/22; C07K 16/244; C07K 2317/31; C07K 2317/34; C07K 2317/522; C07K 2317/526; C07K 2317/55; C07K 2317/565; C07K 2317/622; C07K 2317/92; C07K 2317/94

(57) ABSTRACT

This invention relates to anti-TNFα antibodies and methods of using them in treating diseases and conditions related to TNFα activity, e.g., autoimmune or inflammatory conditions.

### CROSS-REFERENCE TO RELATED APPLICATION

This application claims priority from U.S. Provisional Patent Application 63/356,138, filed Jun. 28, 2022. The disclosure of that priority application is incorporated by reference herein in its entirety.

### SEQUENCE LISTING

The instant application contains a Sequence Listing which has been submitted electronically in XML format and is hereby incorporated by reference in its entirety. The electronic copy of the Sequence Listing, created on Jun. 21, 2023, is named 123314.WO003.xml and is 170,275 bytes in size.

### BACKGROUND OF THE INVENTION

TNFα is a pleiotropic, pro-inflammatory cytokine expressed by cells of the immune system, including monocytes/macrophages (de Waal Malefyt et al., J Exp Med. (1991) 174:1209-20), dendritic cells (DCs) (Ho et al., J Immunol. (2001) 166:1499-506), lymphocytes (Brehm et al., J. Immunol. (2005) 175:5043-49; Fauriat et al., Blood (2010) 115:2167-76; Williamson et al., Proc Natl Acad Sci. USA (1983) 80:5397-401) and neutrophils (Coulthard et al., Clin Exp Immunol. (2012) 170:36-46). It is synthesized as a transmembrane protein on the plasma membrane, and subsequently may be proteolytically processed by TNFα-converting enzyme (TACE), liberating soluble TNFα homotrimer protein (Sedger and McDermott, Cytokine & Growth Factor Reviews (2014) 25:453-72). TNFα is a potent mediator of inflammation and is implicated in the pathogenesis of inflammatory and autoimmune diseases (Kalliolias and Ivashkiv, Nat Rev Rheumatol. (2016) 12:49-62).

TNFα is a well-validated therapeutic target, and multiple TNFα antibodies (infliximab, adalimumab, golimumab, certolizumab) are approved for the treatment of certain rheumatic and inflammatory bowel diseases (IBD). Although the antibodies have dramatically improved the treatment outcome of rheumatic diseases, significant immunogenicity is observed with all four antibodies (van Schouenburg et al., Nat Rev Rheumatol. (2013) 9:164-72). Immunogenicity is associated with lower drug levels, which are associated with discontinuation of treatment, lower efficacy, or treatment failure (Adedokun et al., J. Crohn's Colitis (2017) 11:35-46; Adedokun et al., Inflamm Bowel Dis. (2019) 25:1532-40; Atiqi et al., Frontiers Immunol. (2020) 11:312; Bartelds et al., JAMA (2011) 305:1460-8; Gorovits et al., Clinical & Experimental Immunology (2018) 192:348-65; Jani et al., Ann Rheum Dis (2017) 76:208-13; Kennedy et al., Lancet Gastroenterol. Hepatol. (2019) 4:341-53; Radstake et al., Ann Rheum Dis (2009) 68:1739-45; van Schouenburg et al., supra). Optimization of treatment regimens (e.g., dosage, frequency, co-administration of immunomodulators) diminishes, but does not resolve, the immunogenicity issues (Atiqi et al., supra).

The precise molecular mechanism of the immunogenicity of TNFα antibodies is not clear, and it appears that antibodies directed to TNFα may be inherently prone to generating a greater immune response than antibodies directed to other targets. The FDA-approved TNFα antibodies infliximab (chimeric), certolizumab (humanized), adalimumab (human) and golimumab (human) have varying degrees of protein sequence homology to human antibodies, yet all display significant immunogenicity. By contrast, vedolizumab (anti-α₄□₇) and ustekinumab (anti-IL-12/23), two non-TNFα therapeutic antibodies approved for the treatment of certain rheumatic and inflammatory bowel diseases, bind membrane-associated and soluble targets, respectively, and do not elicit significant immunogenicity (Hanauer et al., J Crohn's Colitis (2019) 14:23-32; Sandborn et al., Gastroenterology (2019) 156: Supplement 1, S-1097, AGA Abstract Tu1718; Van den Berghe et al., J Gastro Hepatol. (2018) 34:1175-81; Wyant et al., J Clin Pharmacol. (2021) 61:1174-81).

Two characteristics of the target protein, TNFα, may contribute to the immunogenicity of the entire class of anti-TNFα antibodies. First, TNFα is expressed as a homotrimer protein, and therefore soluble TNFα can form immune complexes (IC) of varying sizes with antibodies, depending on the relative stoichiometries. Large IC are multivalent lattices of varying antigen-antibody ratios, bind IgG receptors with high avidity, and are internalized into processing pathways that promote cross-presentation of MHC class I and presentation of MHC class II-restricted epitopes (Baker et al., Cell Mol Life Sci (2013) 70:1319-34; Krishna and Nadler, Front Immunol. (2016) 7:21; Weflen et al., Mol Biol Cell (2013) 24:2398-405). Consequently, large IC are potent drivers of immunogenicity, and the pre-formation of IC has long been used as a strategy to drive enhanced immune responses (Terres and Wolins, J Immunol. (1961) 86:361-8; Morrison and Terres, J Immunol. (1966) 96:901-5; Klaus, Immunology (1978) 34:643-52). More recently, a crucial role for IC in immunization against anti-TNFα antibodies in mice was demonstrated (Arnoult et al., J Immunol. (2017) 199:418-24).

The second characteristic of TNFα that potentially contributes to the enhanced immunogenicity of anti-TNFα antibodies is its expression on the plasma membrane of antigen presenting cells of the immune system, including dendritic cells (DC). Membrane-associated TNFα (mTNFα) may allow for the internalization and delivery of TNFα antibodies to the endocytic compartment. Antibody-based targeting of membrane proteins on DC has been exploited as a strategy to induce rapid immune responses (Chen et al., Human Vaccines Immunotherapeutics (2016) 12:612-22; Wang et al., Proc Natl Acad Sci. USA (2000) 96:847-52). Related to this, it was recently demonstrated that antibody bound to mTNFα expressed on dendritic cells was rapidly internalized to the endosomes, trafficked to lysosomes, digested, and the antibody peptides were presented by MHC class II molecules (Deora et al., MABS (2017) 9:680-95). Furthermore, tetanus toxin peptides fused to an anti-TNFα antibody were also presented by DCs, initiating a T cell recall proliferation response (Deora et al., supra).

A less immunogenic TNFα antibody might enable maintenance of more consistent serum antibody levels, have fewer treatment failures, and thus, not require treatment discontinuation or a switch to alternative therapeutic agents.

In view of the critical role of TNFα in the pathogenesis of autoimmune and inflammatory conditions, there is a need for new and improved immune therapies that target TNFα.

### SUMMARY OF THE INVENTION

The present disclosure is directed to novel anti-TNFα antibodies, as well as pharmaceutical compositions comprising one or more of these antibodies, and use of the antibodies and pharmaceutical compositions for treatment of autoimmune and inflammatory conditions. In some embodiments, an antibody of the present disclosure is a variant of a well-characterized, clinically validated anti-TNFα antibody engineered both to enhance its dissociation from TNFα at acidic pH and to prevent the formation of large IC. These characteristics are expected to diminish its trafficking to lysosomes after binding soluble or membrane-associated TNFα, and thus, reduce its immunogenicity. Compared to currently available treatments for autoimmune and inflammatory conditions, including antibody treatments, it is contemplated that the antibodies of the present disclosure may provide a superior clinical response either alone or in combination with another therapeutic for treating autoimmune and/or inflammatory conditions.

In some aspects, the present disclosure provides an anti-TNFα antibody or an antigen-binding portion thereof that binds to the same epitope of human TNFα as a reference antibody comprising:
a) a heavy chain variable domain (VH) that comprises the amino acid sequence of SEQ ID NO: 6 and a light chain variable domain (VL) that comprises the amino acid sequence of SEQ ID NO: 8;
b) a VH that comprises the amino acid sequence of SEQ ID NO: 10 and a VL that comprises the amino acid sequence of SEQ ID NO: 12; or
c) a VH that comprises the amino acid sequence of SEQ ID NO: 14 and a VL that comprises the amino acid sequence of SEQ ID NO: 16;
   wherein said anti-TNFα antibody or antigen-binding portion comprises VH and VL at least 90% identical to the VH and VL of the reference antibody, respectively; and wherein said anti-TNFα antibody or antigen-binding portion has a binding affinity for TNFα that is lower at pH 6.0 than at pH 7.4. In some embodiments, the anti-TNFα antibody or antigen-binding portion may be monovalent.

In some embodiments, the anti-TNFα antibody may comprise
a) a monovalent antigen-binding protein comprising a heavy chain (HC) that comprises a VH at least 90% identical to the VH of the reference antibody and a light chain (LC) that comprises a VL at least 90% identical to the VL of the reference antibody; and
b) a truncated HC lacking the variable domain and CH1 domain;
wherein the antigen-binding protein HC and the truncated HC are capable of dimerization. In certain embodiments, the antigen-binding protein HC and the truncated HC may comprise knobs-into-holes modifications, e.g., wherein the antigen-binding protein HC is of isotype subclass IgG1 and comprises mutations T366S, L368A, and Y407A in the CH3 domain, and the truncated HC is of isotype subclass IgG1 and comprises the mutation T366W in the CH3 domain, wherein the residues are numbered according to the Eu system. Additionally or alternatively, the antigen-binding protein HC may be of isotype subclass IgG1 and comprise the mutation Y349C, and/or the truncated HC may be of isotype subclass IgG1 and comprise the mutation S354C, wherein the residues are numbered according to the Eu system.

The present disclosure also provides an anti-TNFα antibody or antigen-binding portion thereof that comprises heavy chain (HC) CDR1-3 and light chain (LC) CDR1-3 comprising:
a) SEQ ID NOs: 87, 76, 97, 88, 89, 90, respectively;
b) SEQ ID NOs: 87, 76, 98, 88, 89, 90, respectively;
c) SEQ ID NOs: 87, 76, 101, 88, 89, 90, respectively;
d) SEQ ID NOs: 87, 76, 102, 88, 89, 103, respectively;
e) SEQ ID NOs: 87, 76, 77, 104, 89, 90, respectively;
f) SEQ ID NOs: 87, 76, 77, 88, 89, 105, respectively;
g) SEQ ID NOs: 87, 76, 77, 88, 89, 106, respectively;
h) SEQ ID NOs: 87, 76, 77, 107, 89, 90, respectively;
i) SEQ ID NOs: 87, 76, 77, 104, 89, 108, respectively;
j) SEQ ID NOs: 87, 76, 97, 104, 89, and 90, respectively;
k) SEQ ID NOs: 87, 76, 97, 88, 89, and 105, respectively;
l) SEQ ID NOs: 87, 76, 97, 88, 89, and 106, respectively;
m) SEQ ID NOs: 87, 76, 97, 107, 89, and 90, respectively;
n) SEQ ID NOs: 87, 76, 98, 104, 89, and 90, respectively;
o) SEQ ID NOs: 87, 76, 98, 88, 89, and 105, respectively;
p) SEQ ID NOs: 87, 76, 98, 88, 89, and 106, respectively;
q) SEQ ID NOs: 87, 76, 98, 107, 89, and 90, respectively;
r) SEQ ID NOs: 87, 76, 101, 104, 89, and 90, respectively;
s) SEQ ID NOs: 87, 76, 101, 88, 89, and 105, respectively;
t) SEQ ID NOs: 87, 76, 101, 88, 89, and 106, respectively; or
u) SEQ ID NOs: 87, 76, 101, 107, 89, and 90, respectively.

In some embodiments, the antibody or antigen-binding portion comprises a heavy chain variable domain (VH) and a light chain variable domain (VL) that comprise:
a) SEQ ID NOs: 30 and 16, respectively;
b) SEQ ID NOs: 32 and 16, respectively;
c) SEQ ID NOs: 34 and 16, respectively;
d) SEQ ID NOs: 36 and 38, respectively;
e) SEQ ID NOs: 14 and 40, respectively;
f) SEQ ID NOs: 14 and 42, respectively;
g) SEQ ID NOs: 14 and 44, respectively;
h) SEQ ID NOs: 14 and 46, respectively;
i) SEQ ID NOs: 14 and 48, respectively;
j) SEQ ID NOs: 30 and 40, respectively;
k) SEQ ID NOs: 30 and 42, respectively;
l) SEQ ID NOs: 30 and 44, respectively;
m) SEQ ID NOs: 30 and 46, respectively;
n) SEQ ID NOs: 32 and 40, respectively;
o) SEQ ID NOs: 32 and 42, respectively;
p) SEQ ID NOs: 32 and 44, respectively;
q) SEQ ID NOs: 32 and 46, respectively;
r) SEQ ID NOs: 34 and 40, respectively;
s) SEQ ID NOs: 34 and 42, respectively;
t) SEQ ID NOs: 34 and 44, respectively; or
u) SEQ ID NOs: 34 and 46, respectively.

The present disclosure also provides an anti-TNFα antibody or antigen-binding portion thereof that comprises heavy chain (HC) CDR1-3 and light chain (LC) CDR1-3 comprising:
a) SEQ ID NOs: 81, 76, 109, 83, 79, and 80, respectively;
b) SEQ ID NOs: 81, 76, 110, 83, 79, and 80, respectively;
c) SEQ ID NOs: 81, 76, 111, 83, 79, and 80, respectively;
d) SEQ ID NOs: 81, 76, 82, 112, 79, and 80, respectively;
e) SEQ ID NOs: 81, 76, 82, 83, 79, and 99, respectively;
f) SEQ ID NOs: 81, 76, 82, 83, 79, and 113, respectively;
g) SEQ ID NOs: 81, 76, 82, 83, 79, and 100, respectively;
h) SEQ ID NOs: 81, 76, 82, 114, 79, and 80, respectively;
i) SEQ ID NOs: 81, 76, 82, 83, 79, and 115, respectively;
j) SEQ ID NOs: 81, 76, 82, 112, 79, and 116, respectively;
k) SEQ ID NOs: 81, 76, 82, 117, 79, and 80, respectively;
l) SEQ ID NOs: 81, 76, 82, 118, 79, and 80, respectively; or
m) SEQ ID NOs: 81, 76, 82, 119, 79, and 80, respectively.

In some embodiments, the antibody or antigen-binding portion comprises a VH and a VL that comprise:
a) SEQ ID NOs: 50 and 8, respectively;
b) SEQ ID NOs: 52 and 8, respectively;
c) SEQ ID NOs: 54 and 8, respectively;
d) SEQ ID NOs: 6 and 56, respectively;
e) SEQ ID NOs: 6 and 58, respectively;
f) SEQ ID NOs: 6 and 60, respectively;
g) SEQ ID NOs: 6 and 62, respectively;
h) SEQ ID NOs: 6 and 64, respectively;
i) SEQ ID NOs: 6 and 66, respectively;
j) SEQ ID NOs: 6 and 68, respectively;
k) SEQ ID NOs: 6 and 70, respectively;
l) SEQ ID NOs: 6 and 72, respectively; or
m) SEQ ID NOs: 6 and 74, respectively.

In some embodiments, an anti-TNFα antibody or antigen-binding portion of the present disclosure is monovalent. In certain embodiments, the monovalent antibody comprises
a) a monovalent antigen-binding protein that comprises an HC comprising a VH of an antibody described herein and an LC comprising a VL of an antibody described herein; and
b) a truncated HC lacking the variable domain and CH1 domain;
wherein the antigen-binding protein HC and the truncated HC are capable of dimerization.

In particular embodiments, the monovalent antibody may comprise
a) a monovalent antigen-binding protein that comprises an HC comprising a VH with the amino acid sequence of SEQ ID NO: 50 and an LC comprising a VL with the amino acid sequence of SEQ ID NO: 8; and
b) a truncated HC lacking the variable domain and CH1 domain;
wherein the antigen-binding protein HC and the truncated HC are capable of dimerization.

In particular embodiments, the monovalent antibody may comprise
a) a monovalent antigen-binding protein that comprises an HC comprising a VH with the amino acid sequence of SEQ ID NO: 54 and an LC comprising a VL with the amino acid sequence of SEQ ID NO: 8; and
b) a truncated HC lacking the variable domain and CH1 domain;
wherein the antigen-binding protein HC and the truncated HC are capable of dimerization.

In particular embodiments, the monovalent antibody may comprise
a) a monovalent antigen-binding protein that comprises an HC comprising a VH with the amino acid sequence of SEQ ID NO: 6 and an LC comprising a VL with the amino acid sequence of SEQ ID NO: 56; and
b) a truncated HC lacking the variable domain and CH1 domain;
wherein the antigen-binding protein HC and the truncated HC are capable of dimerization.

In particular embodiments, the monovalent antibody may comprise
a) a monovalent antigen-binding protein that comprises an HC comprising a VH with the amino acid sequence of SEQ ID NO: 6 and an LC comprising a VL with the amino acid sequence of SEQ ID NO: 64; and
b) a truncated HC lacking the variable domain and CH1 domain;
wherein the antigen-binding protein HC and the truncated HC are capable of dimerization.

In particular embodiments, the monovalent antibody may comprise
a) a monovalent antigen-binding protein that comprises an HC comprising a VH with the amino acid sequence of SEQ ID NO: 6 and an LC comprising a VL with the amino acid sequence of SEQ ID NO: 68; and
b) a truncated HC lacking the variable domain and CH1 domain;
wherein the antigen-binding protein HC and the truncated HC are capable of dimerization.

In particular embodiments, the monovalent antibody may comprise
a) a monovalent antigen-binding protein that comprises an HC comprising a VH with the amino acid sequence of SEQ ID NO: 14 and an LC comprising a VL with the amino acid sequence of SEQ ID NO: 44; and
b) a truncated HC lacking the variable domain and CH1 domain;
wherein the antigen-binding protein HC and the truncated HC are capable of dimerization.

In some embodiments of the monovalent antibodies in the above-described heterotrimeric format, the antigen-binding protein HC and the truncated HC comprise knobs-into-holes modifications, e.g., wherein the antigen-binding protein HC is of isotype subclass IgG1 and comprises mutations T366S, L368A, and Y407A in the CH3 domain and the truncated HC is of isotype subclass IgG1 and comprises the mutation T366W in the CH3 domain, wherein the residues are numbered according to the Eu system. Additionally or alternatively, the antigen-binding protein HC may be of isotype subclass IgG1 and comprise the mutation Y349C and/or the truncated HC may be of isotype subclass IgG1 and comprise the mutation S354C, wherein the residues are numbered according to the Eu system.

In some embodiments, a monovalent antibody described herein comprises
a) a single-chain variable fragment (scFv) that comprises said VH and said VL, linked to an Fc monomer; and
b) a truncated HC lacking the variable domain and CH1 domain;
wherein the Fc monomer linked to the scFv, and the truncated HC, are capable of dimerization. In certain embodiments, the Fc monomer linked to the scFv, and the truncated HC, comprise knobs-into-holes modifications, e.g., wherein the Fc monomer linked to the scFv is of isotype subclass IgG1 and comprises mutations T366S, L368A, and Y407A in the CH3 domain, and the truncated HC is of isotype subclass IgG1 and comprises the mutation T366W in the CH3 domain, wherein the residues are numbered according to the Eu system. Additionally or alternatively, the Fc monomer linked to the scFv may be of isotype subclass IgG1 and comprise the mutation Y349C, and/or the truncated HC may be of isotype subclass IgG1 and comprise the mutation S354C, wherein the residues are numbered according to the Eu system.

In particular embodiments, an anti-TNFα antibody or antigen-binding portion described herein has a binding affinity for human TNFα that is lower at pH 6.0 than at pH 7.4. In comparison to an antibody comprising VH and VL amino acid sequences of SEQ ID NOs: 2 and 4, respectively; SEQ ID NOs: 6 and 8, respectively; SEQ ID NOs: 10 and 12, respectively; or SEQ ID NOs: 14 and 16, respectively, the antibody or portion may undergo less degradation in vivo, undergo increased recycling to the cell surface in vivo, have a longer half-life in vivo, be less immunogenic in vivo; or any combination thereof; in certain embodiments, the antibody or antigen-binding portion does not form large immune complexes.

The present disclosure also provides a bispecific binding molecule having the binding specificity of an anti-TNFα antibody of the present disclosure and the binding specificity of a second, distinct antibody. In some embodiments, the second antibody is an anti-IL 17A antibody, an anti-IL23 antibody, or an anti-angiopoietin 2 (Ang2) antibody.

The present disclosure also provides an immunoconjugate comprising an anti-TNFα antibody or antigen-binding portion of the present disclosure linked to a therapeutic agent. In some embodiments, the therapeutic agent is an anti-inflammatory or immunosuppressive agent, e.g., a steroid.

The present disclosure also provides isolated nucleic acid molecule(s) comprising nucleotide sequences that encode the heavy and light chain sequences of an anti-TNFα antibody or antigen-binding portion of the present disclosure. In some embodiments, the isolated nucleic acid molecule(s) comprise the nucleotide sequences of:
a) SEQ ID NOs: 29 and 15;
b) SEQ ID NOs: 31 and 15;
c) SEQ ID NOs: 33 and 15;
d) SEQ ID NOs: 35 and 37;
e) SEQ ID NOs: 13 and 39;
f) SEQ ID NOs: 13 and 41;
g) SEQ ID NOs: 13 and 43;
h) SEQ ID NOs: 13 and 45;
i) SEQ ID NOs: 13 and 47;
j) SEQ ID NOs: 29 and 39;
k) SEQ ID NOs: 29 and 41;
l) SEQ ID NOs: 29 and 43;
m) SEQ ID NOs: 29 and 45;
n) SEQ ID NOs: 31 and 39;
o) SEQ ID NOs: 31 and 41;
p) SEQ ID NOs: 31 and 43;
q) SEQ ID NOs: 31 and 45;
r) SEQ ID NOs: 33 and 39;
s) SEQ ID NOs: 33 and 41;
t) SEQ ID NOs: 33 and 43;
u) SEQ ID NOs: 33 and 45;
v) SEQ ID NOs: 49 and 7;
w) SEQ ID NOs: 51 and 7;
x) SEQ ID NOs: 53 and 7;
y) SEQ ID NOs: 5 and 55;
z) SEQ ID NOs: 5 and 57;
aa) SEQ ID NOs: 5 and 59;
bb) SEQ ID NOs: 5 and 61;
cc) SEQ ID NOs: 5 and 63;
dd) SEQ ID NOs: 5 and 65;
ee) SEQ ID NOs: 5 and 67;
ff) SEQ ID NOs: 5 and 69;
gg) SEQ ID NOs: 5 and 71; or
hh) SEQ ID NOs: 5 and 73.

Also provided are vector(s) comprising the isolated nucleic acid molecule(s), wherein the vector(s) further comprise expression control sequence(s) linked operatively to the isolated nucleic acid molecule(s).

The present disclosure also provides a host cell comprising a nucleotide sequence that encodes the heavy chain sequence(s), and a nucleotide sequence that encodes the light chain sequence, of an anti-TNFα antibody or antigen-binding portion of the present disclosure. In some embodiments, the host cell comprises nucleotide sequences selected from a)-hh) above. Also provided is a method for producing an anti-TNFα antibody or an antigen-binding portion thereof, comprising providing the host cell, culturing said host cell under conditions suitable for expression of the antibody or antigen-binding portion, and isolating the resulting antibody or antigen-binding portion.

The present disclosure also provides a pharmaceutical composition comprising an anti-TNFα antibody or antigen-binding portion of the present disclosure, a bispecific binding molecule of the present disclosure, or an immunoconjugate of the present disclosure, and a pharmaceutically acceptable excipient.

The present disclosure also provides a method for treating an autoimmune or inflammatory condition in a patient in need thereof, comprising administering to said patient a therapeutically effective amount of an anti-TNFα antibody or antigen-binding portion of the present disclosure, a bispecific binding molecule of the present disclosure, or an immunoconjugate of the present disclosure.

The present disclosure also provides the use of an anti-TNFα antibody or antigen-binding portion of the present disclosure, a bispecific binding molecule of the present disclosure, or an immunoconjugate of the present disclosure, for the manufacture of a medicament for treating an autoimmune or inflammatory condition in a patient in need thereof.

The present disclosure also provides an anti-TNFα antibody or antigen-binding portion of the present disclosure, a bispecific binding molecule of the present disclosure, or an immunoconjugate of the present disclosure, for use in treating an autoimmune or inflammatory condition in a patient in need thereof.

In some embodiments, the autoimmune or inflammatory condition is rheumatoid arthritis, psoriatic arthritis, plaque psoriasis, ankylosing spondylitis, axial spondyloarthritis, Crohn's disease, ulcerative colitis, hidradenitis suppurativa, polyarticular juvenile idiopathic arthritis, panuveitis, or Alzheimer's disease. In some embodiments, the patient is treated with an additional therapeutic agent, e.g., an anti-inflammatory or immunosuppressive agent, such as methotrexate.

The present disclosure also provides a kit comprising an anti-TNFα antibody or antigen-binding portion of the present disclosure, a bispecific binding molecule of the present disclosure, or an immunoconjugate of the present disclosure. In some embodiments, the kit is for use in a treatment described herein.

The present disclosure also provides an article of manufacture comprising an anti-TNFα antibody or antigen-binding portion of the present disclosure, a bispecific binding molecule of the present disclosure, or an immunoconjugate of the present disclosure, wherein said article of manufacture is suitable for treating an autoimmune or inflammatory condition in a patient in need thereof. In some embodiments, the treatment is a treatment described herein.

Other features, objectives, and advantages of the invention are apparent in the detailed description that follows. It should be understood, however, that the detailed description, while indicating embodiments and embodiments of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a graph depicting the binding of high affinity anti-TNFα Fabs to biotinylated TNFα. Bacterially-expressed Fab was captured on an ELISA plate and subsequently, biotinylated human TNFα was titrated. Following prolonged dissociation at pH 7.4 in the presence of unlabeled 100 nM TNFα, binding of biotinylated TNFα to various Fabs was quantitated. Variants A1, cb1-3, 4.2a-6 and Ab4 all bound more tightly than Ab1 Fab.

FIG. 2 is a pair of graphs depicting the pH sensitivity of binding of high affinity anti-TNFα Fabs. The binding of Fabs to immobilized human TNFα following prolonged dissociation at pH 7.4 (Panel A) or pH 6.0 (Panel B) in the presence of soluble 100 nM TNFα was quantitated.

FIG. 3 is a pair of graphs depicting the pH sensitivity of binding of 4.2a-6 template Fab variants with CDR histidine mutations. The binding of Fab variants to immobilized human TNFα following prolonged dissociation at pH 7.4 (Panel A) or pH 6.0 (Panel B) in the presence of soluble 100 nM TNFα was quantitated.

FIG. 4 is a set of graphs depicting the pH sensitivity of binding of 4.2a-6 template Fab variants with combinatorial CDR histidine mutations. The binding of Fab variants to immobilized human TNFα following prolonged dissociation at pH 7.4 (Panels A and C) or pH 6.0 (Panels B and D) in the presence of soluble 100 nM TNFα was quantitated.

FIG. 5 is a set of graphs depicting the pH sensitivity of binding of A1 template Fab variants with CDR histidine mutations. The binding of Fab variants to immobilized human TNFα following prolonged dissociation at pH 7.4 (Panels A and C) or pH 6.0 (Panels B and D) in the presence of soluble 100 nM TNFα was quantitated.

FIG. 6 is a pair of graphs depicting the pH sensitivity of binding of select A1 template Fab variants with CDR histidine mutations. The binding of Fab variants to immobilized human TNFα following prolonged dissociation at pH 7.4 (Panel A) or pH 6.0 (Panel B) in the presence of soluble 100 nM TNFα was quantitated.

FIG. 7 is a pair of graphs depicting the pH sensitivity of binding of select A1 template or select 4.2a-6 template Fab variants with CDR histidine mutations. The binding of Fab variants to soluble biotinylated murine TNFα following prolonged dissociation at pH 7.4 (Panel A) or pH 6.0 (Panel B) in the presence of soluble 100 nM TNFα was quantitated.

### DETAILED DESCRIPTION OF THE INVENTION

The present disclosure provides novel anti-human TNFα antibodies and antigen-binding portions thereof that can be used to treat autoimmune and/or inflammatory conditions. Unless otherwise stated, as used herein, “TNFα” refers to human TNFα. A human TNFα polypeptide sequence is shown below:

(SEQ ID NO: 122)
MSTESMIRDVELAEEALPKKTGGPQGSRRCLELSLESFLIVAGATTLFC
LLHEGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL

The term “antibody” (Ab) or “immunoglobulin” (Ig), as used herein, may refer to a tetramer comprising two heavy (H) chains (about 50-70 kDa) and two light (L) chains (about 25 kDa) interconnected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. In some embodiments, an antibody described herein may be a bivalent antibody. The term “bivalent antibody,” as used herein, refers to an antibody with two antigen-binding sites. In some embodiments, an antibody described herein may be a monovalent antibody comprising less than two HCs and two LCs (e.g., comprising a single VH and VL, or HC and LC, from an anti-TNFα antibody). The term “monovalent antibody,” as used herein, refers to an antibody with one antigen-binding site.

The assignment of amino acid numbers, and/or of FR and CDR regions, in the heavy or light chain may be in accordance with IMGTR definitions (Lefranc et al., Dev Comp Immunol. (2003) 27(1): 55-77), Eu numbering, or the definitions of Kabat, Sequences of Proteins of Immunological Interest (National Institutes of Health, Bethesda, MD (1987 and 1991); Chothia & Lesk, J Mol Biol. (1987) 196:901-17; Chothia et al., Nature (1989) 342:878-83; MacCallum et al., J Mol Biol. (1996) 262:732-45; or Honegger and Plückthun, J Mol Biol. (2001) 309(3): 657-70 (“AHo” numbering).

In some embodiments, an antibody or antigen-binding portion thereof of the present disclosure is an isolated antibody or antigen-binding portion. The term “isolated protein”, “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between an antigen and an antibody or an antigen-binding fragment thereof, or a related molecule such as a bispecific binding molecule. The intrinsic attractiveness of the antibody for the antigen is typically expressed as the binding affinity equilibrium constant (K_{D}) of a particular antibody-antigen interaction. An antibody or antigen-binding portion is said to specifically bind to an antigen when the K_{D} is ≤1 μM, e.g., ≤100 nM or ≤10 nM. A K_{D} binding affinity constant can be measured, e.g., by surface plasmon resonance (BIAcore™) or Bio-Layer Interferometry, for example using the IBIS MX96 SPR system from IBIS Technologies, the Carterra LSA SPR platform, or the Octet™ system from ForteBio.

The term “epitope” as used herein refers to a portion (determinant) of an antigen that specifically binds to an antibody or an antigen-binding portion thereof. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., TNFα) or a relevant portion thereof, then screening for binding to the epitope.

One can determine whether an antibody binds to the same epitope of TNFα as or competes for binding with an antibody described herein by using methods known in the art, including, without limitation, competition assays, epitope binning, and alanine scanning. In some embodiments, one allows an antibody described herein to bind to TNFα under saturating conditions, and then measures the ability of the test antibody to bind to said antigen. If the test antibody is able to bind to said antigen at the same time as the reference antibody, then the test antibody binds to a different epitope than the reference antibody. However, if the test antibody is not able to bind to the antigen at the same time, then the test antibody binds to the same epitope, an overlapping epitope, or an epitope that is in close proximity to the epitope bound by the antibody described herein. This experiment can be performed using, e.g., ELISA, RIA, BIACORE™, SPR, Bio-Layer Interferometry or flow cytometry. To test whether an antibody described herein cross-competes with another antibody for binding to TNFα, one may use the competition method described above in two directions, i.e., determining if the known antibody blocks the test antibody and vice versa. Such cross-competition experiments may be performed, e.g., using an IBIS MX96 SPR instrument or the Octet™ system.

The term “antigen-binding portion” or “antigen-binding fragment” of an antibody, as used herein, refers to one or more portions or fragments of an antibody that retain the ability to specifically bind to an antigen (e.g., human TNFα, or a portion thereof). It has been shown that certain fragments of a full-length antibody can perform the antigen-binding function of the antibody. Examples of binding fragments encompassed within the term “antigen-binding portion” include (i) a Fab fragment: a monovalent fragment consisting of the VL, VH, CL and CH1 domains; (ii) a F(ab′)2 fragment: a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR) capable of specifically binding to an antigen. Furthermore, although the two domains of the Fv fragment, VL and VH, are encoded by separate genes, they can be joined, using recombinant methods, by a synthetic linker that enables them to be made as a single protein chain in which the VL and VH domains pair to form monovalent molecules known as single chain variable fragments (scFvs). Also within the present disclosure are antigen-binding molecules comprising a VH and/or a VL. In the case of a VH, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites. The present disclosure also contemplates antigen-binding portions of the anti-TNFα antibodies described herein, wherein the antigen-binding portions retain the functional properties of the cognate antibodies. Such antigen-binding portions may be used where the cognate antibody is used.

### Anti-TNFα Antibodies

The present disclosure provides novel therapeutic anti-TNFα antibodies engineered to be less immunogenic. Such engineered antibodies may maintain more consistent serum antibody levels and have greater or more prolonged therapeutic efficacy compared to the parent antibodies. In some embodiments, the antibodies of the present disclosure are engineered to prevent the formation of large immune complexes (IC), to enhance their dissociation from TNFα at acidic pH, or both. As used herein, “large IC” refers to immune complexes that comprise ≥2 TNFα trimers and ≥3 antibodies or antigen-binding portions. In certain embodiments, the antibodies have a pH-sensitive antigen binding function (“pH switch”). The pH switch allows the antibody to bind and neutralize serum (soluble) and membrane-associated TNFα at physiological pH (e.g., ˜pH 7.4), while also enabling dissociation following internalization into the acidic endosomal environment (e.g., ˜pH 6.0). The dissociated antibody may then be recycled to the cell surface via the FcRn, while the antigen is trafficked to the lysosomes for degradation. In certain embodiments, the antibodies are monovalent. Without wishing to be bound by theory, it is contemplated that monovalency reduces or eliminates the formation of large IC via antibody-mediated cross-linking of TNFα. In particular embodiments, the antibodies of the present disclosure incorporate a pH switch and are monovalent.

In some embodiments, an anti-TNFα antibody or antigen-binding portion thereof of the present disclosure is derived from a higher affinity variant of parent anti-TNFα antibody “Ab1,” which comprises the amino acid sequences shown below (variable domains italicized, CDRs underlined):

Ab1 HC
(SEQ ID NO: 120)
EVQLVESGGGLVQPGRSLRLSCAASGFTFDDYAMHWVRQAPGKGLEWVSAITWNSGHIDYAD
SVEGRFTISRDNAKNSLYLQMNSLRAEDTAVYYCAKVSYLSTASSLDYWGQGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
Ab1 LC
(SEQ ID NO: 121)
DIQMTQSPSSLSASVGDRVTITCRASQGIRNYLAWYQQKPGKAPKLLIYAASTLQSGVPSRF
SGSGSGTDFTLTISSLQPEDVATYYCQRYNRAPYTFGQGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

In some embodiments, the higher affinity variant of Ab1 comprises:
a) a heavy chain variable domain (VH) that comprises the amino acid sequence of SEQ ID NO: 6 and a light chain variable domain (VL) that comprises the amino acid sequence of SEQ ID NO: 8 (“A1”);
b) a VH that comprises the amino acid sequence of SEQ ID NO: 10 and a VL that comprises the amino acid sequence of SEQ ID NO: 12 (“cb1-3”); or
c) a VH that comprises the amino acid sequence of SEQ ID NO: 14 and a VL that comprises the amino acid sequence of SEQ ID NO: 16 (“4.2a-6”).

In some embodiments, an anti-TNFα antibody or antigen-binding portion thereof of the present disclosure binds to the same epitope of human TNFα as the reference higher affinity variant, and comprises VH and VL at least 90% identical to the VH and VL, respectively, of the reference higher affinity variant. In some embodiments, the anti-TNFα antibody or antigen-binding portion has VH and VL amino acid sequences that comprise, in total, at least one, two, three, four, or five amino acid substitutions from the VH and VL amino acid sequences of the reference higher affinity variant. In certain embodiments, the VH and VL amino acid sequences comprise, in total, one amino acid substitution from the VH and VL amino acid sequences of the reference higher affinity variant. In certain embodiments, the VH and VL amino acid sequences comprise, in total, two amino acid substitutions from the VH and VL amino acid sequences of the reference higher affinity variant. In certain embodiments, the VH and VL amino acid sequences comprise, in total, three amino acid substitutions from the VH and VL amino acid sequences of the reference higher affinity variant. In certain embodiments, the VH and VL amino acid sequences comprise, in total, four amino acid substitutions from the VH and VL amino acid sequences of the reference higher affinity variant. In certain embodiments, the VH and VL amino acid sequences comprise, in total, five amino acid substitutions from the VH and VL amino acid sequences of the reference higher affinity variant. In particular embodiments, the amino acid substitutions may alter the binding affinity of the antibody or portion at certain pH values; for example, the altered antibody or portion may have a binding affinity for TNFα that is reduced at a lower pH (e.g., pH 6.0) compared to a higher pH (e.g., pH 7.4). In some embodiments, the EC50 for binding at the lower pH may be increased by at least 2-, 5-, 10-, 15-, 20-, 25-, 30-, 50-, 75-, 100-, 500-, 1000-, 2000-, or 4000-fold compared to the binding affinity at the higher pH.

In some embodiments, the amino acid substitution(s) are in the FRs, or the FRs and the CDRs, of the anti-TNFα antibody or antigen-binding portion. In some embodiments, the amino acid substitution(s) are in the CDRs of the anti-TNFα antibody or antigen-binding portion. In certain embodiments, the amino acid substitution(s) are in H-CDR3, L-CDR1, L-CDR3, or any combination thereof (e.g., L-CDR1 and L-CDR3, H-CDR3 and L-CDR1, H-CDR3 and L-CDR3, or H-CDR3, L-CDR1, and L-CDR3). The CDRs may be delineated by the Kabat, Chothia, IMGT, contact, or AHo method, or any combination thereof. In particular embodiments, the CDRs are delineated as shown in the Ab1 sequences above (SEQ ID NOs: 120 and 121).

In certain embodiments, the anti-TNFα antibody or portion comprises
an H-CDR1 comprising a sequence selected from SEQ ID NOs: 75, 81, and 87;
an H-CDR2 comprising SEQ ID NO: 76;
an H-CDR3 comprising a sequence selected from SEQ ID NOs: 77, 82, 97, 98, 101, 102, 109, 110, and 111;
an L-CDR1 comprising a sequence selected from SEQ ID NOs: 78, 83, 84, 88, 104, 107, 112, 114, 117, 118, and 119;
an L-CDR2 comprising a sequence selected from SEQ ID NOs: 79, 85, and 89; and
an L-CDR3 comprising a sequence selected from SEQ ID NOs: 80, 86, 90, 99, 100, 103, 105, 106, 108, 113, 115, and 116;
   wherein the antibody or portion does not comprise the six CDR sequences of SEQ ID NOs: 75, 76, 77, 78, 79, and 80; SEQ ID NOs: 81, 76, 82, 83, 79, and 80; SEQ ID NOs: 81, 76, 77, 84, 85, and 86; or SEQ ID NOs: 87, 76, 77, 88, 89, and 90.

In certain embodiments, the anti-TNFα antibody or portion comprises
a VH comprising a sequence selected from SEQ ID NOs: 2, 6, 10, 14, 30, 32, 34, 36, 50, 52, and 54, or a sequence at least 99%, 91%, 92%, 93%, 94%, 95%, 96%, 97%, 98%, or 99% identical thereto; and
a VL comprising a sequence selected from SEQ ID NOs: 4, 8, 12, 16, 38, 40, 42, 44, 46, 48, 56, 58, 60, 62, 64, 66, 68, 70, 72, and 74, or a sequence at least 99%, 91%, 92%, 93%, 94%, 95%, 96%, 97%, 98%, or 99% identical thereto;
   wherein the antibody or portion does not comprise VH and VL sequences of SEQ ID NOs: 2 and 4, respectively; SEQ ID NOs: 6 and 8, respectively; SEQ ID NOs: 10 and 12, respectively; or SEQ ID NOs: 14 and 16, respectively.

In certain embodiments, the anti-TNFα antibody or portion comprises
an H-CDR1 comprising SEQ ID NO: 87;
an H-CDR2 comprising SEQ ID NO: 76;
an H-CDR3 comprising a sequence selected from SEQ ID NOs: 77, 97, 98, 101, and 102;
an L-CDR1 comprising a sequence selected from SEQ ID NOs: 88, 104, 107;
an L-CDR2 comprising SEQ ID NO: 89; and
an L-CDR3 comprising a sequence selected from SEQ ID NOs: 90, 103, 105, 106, and 108;
   wherein the antibody or portion does not comprise the H-CDR1-3 and L-CDR1-3 sequences of SEQ ID NOs: 87, 76, 77, 88, 89, and 90.

In certain embodiments, the anti-TNFα antibody or portion comprises

H-CDR1-3 sequences comprising
SEQ ID NOs: 87, 76, and 77, respectively;
SEQ ID NOs: 87, 76, and 97, respectively;
SEQ ID NOs: 87, 76, and 98, respectively;
SEQ ID NOs: 87, 76, and 101, respectively; or
SEQ ID NOs: 87, 76, and 102, respectively; and
   L-CDR1-3 sequences comprising
SEQ ID NOs: 88, 89, and 90, respectively;
SEQ ID NOs: 88, 89, and 103, respectively;
SEQ ID NOs: 104, 89, and 90, respectively;
SEQ ID NOs: 88, 89, and 105, respectively;
SEQ ID NOs: 88, 89, and 106, respectively;
SEQ ID NOs: 107, 89, and 90, respectively; or
SEQ ID NOs: 104, 89, and 108, respectively;
   wherein the antibody or portion does not comprise the H-CDR1-3 and L-CDR1-3 sequences of SEQ ID NOs: 87, 76, 77, 88, 89, and 90, respectively.

In certain embodiments, the anti-TNFα antibody or portion comprises H-CDR1-3 and L-CDR1-3 sequences comprising
SEQ ID NOs: 87, 76, 97, 88, 89, 90, respectively;
SEQ ID NOs: 87, 76, 98, 88, 89, 90, respectively;
SEQ ID NOs: 87, 76, 101, 88, 89, 90, respectively;
SEQ ID NOs: 87, 76, 102, 88, 89, 103, respectively;
SEQ ID NOs: 87, 76, 77, 104, 89, 90, respectively;
SEQ ID NOs: 87, 76, 77, 88, 89, 105, respectively;
SEQ ID NOs: 87, 76, 77, 88, 89, 106, respectively;
SEQ ID NOs: 87, 76, 77, 107, 89, 90, respectively;
SEQ ID NOs: 87, 76, 77, 104, 89, 108, respectively;
SEQ ID NOs: 87, 76, 97, 104, 89, and 90, respectively;
SEQ ID NOs: 87, 76, 97, 88, 89, and 105, respectively;
SEQ ID NOs: 87, 76, 97, 88, 89, and 106, respectively;
SEQ ID NOs: 87, 76, 97, 107, 89, and 90, respectively;
SEQ ID NOs: 87, 76, 98, 104, 89, and 90, respectively;
SEQ ID NOs: 87, 76, 98, 88, 89, and 105, respectively;
SEQ ID NOs: 87, 76, 98, 88, 89, and 106, respectively;
SEQ ID NOs: 87, 76, 98, 107, 89, and 90, respectively;
SEQ ID NOs: 87, 76, 101, 104, 89, and 90, respectively;
SEQ ID NOs: 87, 76, 101, 88, 89, and 105, respectively;
SEQ ID NOs: 87, 76, 101, 88, 89, and 106, respectively; or
SEQ ID NOs: 87, 76, 101, 107, 89, and 90, respectively.

In certain embodiments, the anti-TNFα antibody or portion comprises a VH comprising a sequence selected from SEQ ID NOs: 14, 30, 32, 34, and 36, or a sequence at least 99%, 91%, 92%, 93%, 94%, 95%, 96%, 97%, 98%, or 99% identical thereto; and a VL comprising a sequence selected from SEQ ID NOs: 16, 38, 40, 42, 44, 46, and 48, or a sequence at least 99%, 91%, 92%, 93%, 94%, 95%, 96%, 97%, 98%, or 99% identical thereto; wherein the antibody or portion does not comprise the VH and VL sequences of SEQ ID NOs: 14 and 16, respectively.

In certain embodiments, the anti-TNFα antibody or portion comprises a VH and a VL comprising the sequences of
SEQ ID NOs: 30 and 16, respectively;
SEQ ID NOs: 32 and 16, respectively;
SEQ ID NOs: 34 and 16, respectively;
SEQ ID NOs: 36 and 38, respectively;
SEQ ID NOs: 14 and 40, respectively;
SEQ ID NOs: 14 and 42, respectively;
SEQ ID NOs: 14 and 44, respectively;
SEQ ID NOs: 14 and 46, respectively;
SEQ ID NOs: 14 and 48, respectively;
SEQ ID NOs: 30 and 40, respectively;
SEQ ID NOs: 30 and 42, respectively;
SEQ ID NOs: 30 and 44, respectively;
SEQ ID NOs: 30 and 46, respectively;
SEQ ID NOs: 32 and 40, respectively;
SEQ ID NOs: 32 and 42, respectively;
SEQ ID NOs: 32 and 44, respectively;
SEQ ID NOs: 32 and 46, respectively;
SEQ ID NOs: 34 and 40, respectively;
SEQ ID NOs: 34 and 42, respectively;
SEQ ID NOs: 34 and 44, respectively; or
SEQ ID NOs: 34 and 46, respectively.

In certain embodiments, the anti-TNFα antibody or portion comprises
an H-CDR1 comprising SEQ ID NO: 81;
an H-CDR2 comprising SEQ ID NO: 76;
an H-CDR3 comprising a sequence selected from SEQ ID NOs: 82, 109, 110, and 111;
an L-CDR1 comprising a sequence selected from SEQ ID NOs: 83, 112, 114, 117, 118, and 119;
an L-CDR2 comprising SEQ ID NO: 79; and
an L-CDR3 comprising a sequence selected from SEQ ID NOs: 80, 99, 100, 113, 115, and 116;
   wherein the antibody or portion does not comprise the H-CDR1-3 and L-CDR1-3 sequences of SEQ ID NOs: 81, 76, 82, 83, 79, and 80, respectively.

In certain embodiments, the anti-TNFα antibody or portion comprises

H-CDR1-3 sequences comprising
SEQ ID NOs: 81, 76, and 82, respectively;
SEQ ID NOs: 81, 76, and 109, respectively;
SEQ ID NOs: 81, 76, and 110, respectively; or
SEQ ID NOs: 81, 76, and 111, respectively; and
   L-CDR1-3 sequences comprising
SEQ ID NOs: 83, 79, and 80, respectively;
SEQ ID NOs: 112, 79, and 80, respectively;
SEQ ID NOs: 83, 79, and 99, respectively;
SEQ ID NOs: 83, 79, and 113, respectively;
SEQ ID NOs: 83, 79, and 100, respectively;
SEQ ID NOs: 114, 79, and 80, respectively;
SEQ ID NOs: 83, 79, and 115, respectively;
SEQ ID NOs: 112, 79, and 116, respectively;
SEQ ID NOs: 117, 79, and 80, respectively;
SEQ ID NOs: 118, 79, and 80, respectively; or
SEQ ID NOs: 119, 79, and 80, respectively;
   wherein the antibody or portion does not comprise the H-CDR1-3 and L-CDR1-3 sequences of SEQ ID NOs: 81, 76, 82, 83, 79, and 80, respectively.

In certain embodiments, the anti-TNFα antibody or portion comprises H-CDR1-3 and L-CDR1-3 sequences comprising
SEQ ID NOs: 81, 76, 109, 83, 79, and 80, respectively;
SEQ ID NOs: 81, 76, 110, 83, 79, and 80, respectively;
SEQ ID NOs: 81, 76, 111, 83, 79, and 80, respectively;
SEQ ID NOs: 81, 76, 82, 112, 79, and 80, respectively;
SEQ ID NOs: 81, 76, 82, 83, 79, and 99, respectively;
SEQ ID NOs: 81, 76, 82, 83, 79, and 113, respectively;
SEQ ID NOs: 81, 76, 82, 83, 79, and 100, respectively;
SEQ ID NOs: 81, 76, 82, 114, 79, and 80, respectively;
SEQ ID NOs: 81, 76, 82, 83, 79, and 115, respectively;
SEQ ID NOs: 81, 76, 82, 112, 79, and 116, respectively;
SEQ ID NOs: 81, 76, 82, 117, 79, and 80, respectively;
SEQ ID NOs: 81, 76, 82, 118, 79, and 80, respectively; or
SEQ ID NOs: 81, 76, 82, 119, 79, and 80, respectively.

In certain embodiments, the anti-TNFα antibody or portion comprises a VH comprising a sequence selected from SEQ ID NOs: 6, 50, 52, and 54, or a sequence at least 99%, 91%, 92%, 93%, 94%, 95%, 96%, 97%, 98%, or 99% identical thereto; and a VL comprising a sequence selected from SEQ ID NOs: 8, 56, 58, 60, 62, 64, 66, 68, 70, 72, and 74, or a sequence at least 99%, 91%, 92%, 93%, 94%, 95%, 96%, 97%, 98%, or 99% identical thereto; wherein the antibody or portion does not comprise the VH and VL sequences of SEQ ID NOs: 6 and 8, respectively.

In certain embodiments, the anti-TNFα antibody or portion comprises a VH and a VL comprising the sequences of
SEQ ID NOs: 50 and 8, respectively;
SEQ ID NOs: 52 and 8, respectively;
SEQ ID NOs: 54 and 8, respectively;
SEQ ID NOs: 6 and 56, respectively;
SEQ ID NOs: 6 and 58, respectively;
SEQ ID NOs: 6 and 60, respectively;
SEQ ID NOs: 6 and 62, respectively;
SEQ ID NOs: 6 and 64, respectively;
SEQ ID NOs: 6 and 66, respectively;
SEQ ID NOs: 6 and 68, respectively;
SEQ ID NOs: 6 and 70, respectively;
SEQ ID NOs: 6 and 72, respectively; or
SEQ ID NOs: 6 and 74, respectively.

An anti-TNFα antibody described herein can be an IgG, an IgM, an IgE, an IgA, or an IgD molecule, but is typically of the IgG isotype, e.g., of IgG subclass IgG1, IgG2a or IgG2b, IgG3, or IgG4. In particular embodiments, the antibody is of the isotype subclass IgG1.

An anti-TNFα antibody described herein may be monovalent/in a monomeric format. Examples of such formats include any format comprising a single antigen-binding domain (e.g., a single VH/VL pair), including Fab, scFv, single domain antibody, VHH/nanobody, UniDab, VNAR etc. Also contemplated are monovalent forms of binding molecules such as adnexins, affibodies, affilins, anticalins, avimers, and DARPins, wherein the binding molecules have the binding specificity of an anti-TNFα antibody described herein. A monovalent antibody described herein may comprise a constant (Fc) region component (e.g., a full Fc region) that provides effector function (e.g., full effector function).

In certain embodiments, an anti-TNFα antibody described herein comprises an antigen-binding protein, which may be monovalent, bivalent, or multivalent. In some embodiments, the antigen-binding protein is monovalent (also termed a “Fab” herein) and comprises a VH and a VL, or an HC and an LC, of an anti-TNFα antibody described herein. In particular embodiments, the antigen-binding protein is monovalent and comprises an HC and an LC of an anti-TNFα antibody described herein. In some embodiments, a monovalent anti-TNFα antibody described herein is a heterotrimer comprising an antibody HC coupled to an antibody LC to form an antigen-binding domain, wherein the antibody HC dimerizes with a polypeptide that is a “truncated heavy chain” (i.e., an HC lacking the variable and CH1 domains) to form an Fc domain. The truncated heavy chain may comprise or consist of an Fc monomer (i.e., one of two polypeptides that dimerize to form an Fc domain). In certain embodiments, the Fc monomer comprises CH2 and CH3 of an antibody heavy chain such as an IgG heavy chain; the IgG may be IgG1, IgG2, IgG2, or IgG4. In particular embodiments, dimerization between the antibody HC and the truncated HC provides a fully functional Fc domain, which may preserve the pharmacokinetic and effector function properties of the parent antibody (e.g., Ab1 or a higher affinity variant thereof as described herein).

In certain embodiments, a monovalent anti-TNFα antibody described herein comprises an scFv. In certain embodiments, the scFv comprises a VH and a VL of an anti-TNFα antibody described herein. In particular embodiments, the monovalent anti-TNFα antibody described herein is a heterodimer (e.g., a single chain comprising an scFv and Fc monomer of an anti-TNFα antibody described herein, and an additional (truncated) HC lacking the variable domain and CH1 domain (e.g., a constant domain fragment such as an Fc monomer). The single chain may be arranged, for example, as VL-linker-VH-Fc monomer. In particular embodiments, dimerization between the Fc monomer portion of the single chain and the Fc monomer portion of the additional HC provides a fully functional Fc domain, which may preserve the pharmacokinetic and effector function properties of the parent antibody (e.g., Ab1 or a higher affinity variant thereof as described herein).

In certain embodiments of the monovalent anti-TNFα antibody heterotrimer or heterodimer, the heavy chain Fc heterodimer is, e.g., in a format described in Brinkmann and Kontermann, MAbs 9:182-212 (2017). For example, a “knobs-into-holes,” HA-TF, ZW1, CH3 charge pair, EW-RVT, LUZ-Y, Strand Exchange Engineered Domain body (SEEDbody), Biclonic, DuoBody, BEAT, 7.8.60, 20.8.34, Triomab/Quadroma, or CrossMAb strategy may be used to promote heterodimerization (e.g., over homodimerization) of the antibody heavy chain Fc monomer and the truncated heavy chain Fc monomer. In certain embodiments, a “knobs-into-holes” approach may be used, wherein a “knob” variant of a domain is obtained by replacing an amino acid with a small side chain (for example, alanine, asparagine, aspartic acid, glycine, serine, threonine or valine) with another amino acid with a larger side chain (for example, arginine, phenylalanine, tyrosine, or tryptophan). A “hole” variant of a domain is obtained by replacing an amino acid with a large side chain (for example, arginine, phenylalanine, tyrosine, or tryptophan) with another amino acid with a smaller side chain (for example, alanine, asparagine, aspartic acid, glycine, serine, threonine or valine). In certain embodiments, the knob and/or hole mutations are in the CH3 domain. In particular embodiments, both Fc monomers are derived from IgG1, and the antibody heavy chain Fc monomer may comprise mutations T366S, L368A, and Y407A in the CH3 domain and the truncated heavy chain Fc monomer may comprise the mutation T366W in the CH3 domain, or vice-versa, wherein the residues are numbered according to the Eu system. Additionally or alternatively, the antibody heavy chain Fc monomer may comprise the mutation Y349C and the truncated heavy chain Fc monomer may comprise the mutation S354C, or vice-versa, wherein the residues are numbered according to the Eu system.

In some embodiments, the present disclosure provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 81, 76, 109, 83, 79, and 80, respectively. In some embodiments, the antibody or portion comprises a VH comprising SEQ ID NO: 50 and a VL comprising SEQ ID NO: 8. In certain embodiments, the antibody is monovalent and comprises a monovalent antigen-binding protein comprising an HC and an LC with said VH and VL, respectively, and a truncated HC lacking the variable domain and CH1 domain, wherein the antigen-binding protein HC and the truncated HC are capable of dimerization.

In some embodiments, the present disclosure provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 81, 76, 111, 83, 79, and 80, respectively. In some embodiments, the antibody or portion comprises a VH comprising SEQ ID NO: 54 and a VL comprising SEQ ID NO: 8. In certain embodiments, the antibody is monovalent and comprises a monovalent antigen-binding protein comprising an HC and an LC with said VH and VL, respectively, and a truncated HC lacking the variable domain and CH1 domain, wherein the antigen-binding protein HC and the truncated HC are capable of dimerization.

In some embodiments, the present disclosure provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 81, 76, 82, 112, 79, and 80, respectively. In some embodiments, the antibody or portion comprises a VH comprising SEQ ID NO: 6 and a VL comprising SEQ ID NO: 56. In certain embodiments, the antibody is monovalent and comprises a monovalent antigen-binding protein comprising an HC and an LC with said VH and VL, respectively, and a truncated HC lacking the variable domain and CH1 domain, wherein the antigen-binding protein HC and the truncated HC are capable of dimerization.

In some embodiments, the present disclosure provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 81, 76, 82, 114, 79, and 80, respectively. In some embodiments, the antibody or portion comprises a VH comprising SEQ ID NO: 6 and a VL comprising SEQ ID NO: 64. In certain embodiments, the antibody is monovalent and comprises a monovalent antigen-binding protein comprising an HC and an LC with said VH and VL, respectively, and a truncated HC lacking the variable domain and CH1 domain, wherein the antigen-binding protein HC and the truncated HC are capable of dimerization.

In some embodiments, the present disclosure provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 81, 76, 82, 112, 79, and 116, respectively. In some embodiments, the antibody or portion comprises a VH comprising SEQ ID NO: 6 and a VL comprising SEQ ID NO: 68. In certain embodiments, the antibody is monovalent and comprises a monovalent antigen-binding protein comprising an HC and an LC with said VH and VL, respectively, and a truncated HC lacking the variable domain and CH1 domain, wherein the Fab HC and the truncated HC are capable of dimerization.

In some embodiments, the present disclosure provides an anti-TNFα antibody or an antigen-binding portion thereof (e.g., a monovalent anti-TNFα antibody or an antigen-binding portion thereof), wherein said antibody comprises H-CDR1-3 and L-CDR1-3 that comprise SEQ ID NOs: 87, 76, 77, 88, 89, and 106, respectively. In some embodiments, the antibody or portion comprises a VH comprising SEQ ID NO: 14 and a VL comprising SEQ ID NO: 44. In certain embodiments, the antibody is monovalent and comprises a monovalent antigen-binding protein comprising an HC and an LC with said VH and VL, respectively, and a truncated HC lacking the variable domain and CH1 domain, wherein the Fab HC and the truncated HC are capable of dimerization.

In some embodiments, the constant region(s) of an anti-TNFα antibody or antigen-binding portion thereof described herein are mutated, e.g., to increase the effector function of the antibody or antigen-binding portion (e.g., as described in Wang et al., Protein Cell (2018) 9(1):63-73; Kellner et al., Transfus Med Hemother. (2017) 44:327-36; or Robkopf et al., Antibodies (2020) 9(4): 63). In certain embodiments, the mutations enhance ADCC or CDC. In some embodiments, the mutations are in an IgG1 and comprise (Eu numbering) L235V, G236A, S239D, F243L, S267E, H268F, R292P, S298A, Y300L, V305I, S324T, N325S, K326W, L328F, A330L, 1332E, E333A, E333S, K334A, P396L, or any combination thereof. For example, the mutations may comprise F243L/R292P/Y300L/V305I/P396L; S239D/1332E; S239D/1332E/A330L; S298A/E333A/K334A; L235V/F143L/R292P/Y300L/P396L; G236A/S239D/1332E; K326W/E333S; S267E/H268F/S324T; S267E/H268F/S324T/G236A/1332E; S267E/L328F; or N325S/L328F. In some embodiments, the mutations may comprise L234Y/L235Q/G236W/S239M/H268D/D270E/S298A on one heavy chain and D270E/K326D/A330M/K334E on the other heavy chain.

Additionally or alternatively, the constant region(s) of an anti-TNFα antibody or antigen-binding portion thereof described herein may be mutated to prolong the half-life of the antibody or portion (e.g., as described in Maeda et al., MAbs (2017) 9(5): 844-53; Wang et al., supra; or PCT Patent Publication WO 00/09560). In some embodiments, the mutations are in an IgG1 and comprise (Eu numbering) M252Y, S254T, T256E, M428L N434A, N434S, Y436T, Y436V, Q438R, S440E, or any combination thereof. For example, the mutations may comprise M252Y/S254T/T256E, M428L/N434S, N434A/Y436T/Q438R/S440E; N434A/Y436V/Q438R/S440E; M428L/N434A/Y436T/Q438R/S440E; M428L/N434A/Y436V/Q438R/S440E; or M428L/N434A/Q438R/S440E.

In some embodiments, the antibody is glycoengineered to enhance effector function (e.g., as described in Li et al., Proc Natl Acad Sci USA (2017) 114(13): 3485-90; or Robkopf et al., supra). In certain embodiments, the antibody is glycoengineered to reduce fucose (e.g., afucosylated variants) or sialic acid content or through GlycoMAb™ technology.

In some embodiments, the framework or constant region(s) of an anti-TNFα antibody or antigen-binding portion thereof described herein are mutated to alter the immunogenicity of the antibody, and/or to provide a site for covalent or non-covalent binding to another molecule.

Any combination of the mutations described herein is also contemplated.

In some embodiments, an anti-TNFα antibody or antigen-binding portion of the present disclosure may, e.g., bind to human TNFα with an EC50 of no more than 1e-007 M, 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M, e.g., at pH 7.4. In certain embodiments, binding of the antibody or antigen-binding portion to human TNFα is reduced by at least 2-, 5-, 10-, 15-, 20-, 25-, 30-, 100-, 500-, 1000-, 1500-, 2000-, 2500-, 3000-, or 4000-fold at pH 6.0. In particular embodiments, the antibody or antigen-binding portion has a dissociation rate at pH 6.0 that is at least 20-, 30-, 40-, 50-, 75-, 100-, 150-, 200-, 250-, 300-, 400-, 500-, 600-, 700-, 800-, 900-, 1000-, 1500-, 2000-, or 2500-fold faster than that of Ab1 or monovalent Ab1, or a higher affinity variant thereof as described herein. In some embodiments, the antibody or antigen-binding portion binds to human TNFα with an EC50 of no more than 50 nM at pH 7.4 and has a dissociation rate for human TNFα at pH 6.0 that is at least 10-fold, 100-fold, or 1000-fold greater than the dissociation rate of Ab1 or monovalent Ab1, or a higher affinity variant thereof as described herein. In some embodiments, the antibody or antigen-binding portion binds to human TNFα with an EC50 of no more than 50 or 100 nM at pH 7.4 and has a dissociation rate for human TNFα of greater than 2e-004 s-1 at pH 6.0. In some embodiments, the antibody or antigen-binding portion binds to human TNFα with higher affinity at pH 7.4 than monovalent antibody AF-M2631 (comprising VH and VL sequences of SEQ ID NOs: 22 and 4, respectively) and/or AF-M2637 (comprising VH and VL sequences of SEQ ID NOs: 2 and 28, respectively). In certain embodiments, the antibody or antigen-binding portion binds to human TNFα with higher affinity at pH 7.4 than monovalent antibody AF-M2637.

In some embodiments, an anti-TNFα antibody or antigen-binding portion of the present disclosure binds to murine TNFα with an EC50 of no more than 1e-006 M, 5e-007 M, 1e-007 M, 5e-008 M, 2e-008 M, 1e-008 M, 5e-009 M, 2e-009 M, 1e-009 M, 5e-010 M, 2e-010 M, 1e-011 M, 5e-011 M, 2e-011 M, 1e-011 M, 5e-012 M, 2e-012 M, or 1e-012 M, e.g., at pH 7.4. In certain embodiments, binding of the antibody or antigen-binding portion to murine TNFα is reduced by at least 2-, 5-, 10-, 15-, 20-, 25-, 30-, 100-, 500-, 1000-, 1500-, 2000-, 2500-, 3000-, or 4000-fold at pH 6.0. In particular embodiments, the antibody or antigen-binding portion has a dissociation rate at pH 6.0 that is at least 20-, 30-, 40-, 50-, 75-, 100-, 150-, 200-, 250-, 300-, 400-, 500-, 600-, 700-, 800-, 900-, 1000-, 1500-, 2000-, or 2500-fold faster than that of Ab1 or a higher affinity variant thereof as described herein.

In certain embodiments, an anti-TNFα antibody or antigen-binding portion of the present disclosure binds to human and murine TNFα, for example with an EC50 of no more than 1e-008 M, 5e-009 M, 2e-009M, 1e-009M, 5e-010 M, 2e-010 M, 1e-011 M, 5e-011 M, 2e-011 M, 1e-011 M, 5e-012 M, 2e-012 M, or 1e-012 M, or any combination thereof, for each antigen, e.g., at pH 7.4.

In some embodiments, an anti-TNFα antibody or antigen-binding portion of the present disclosure has a longer half-life in vivo than Ab1 or a higher affinity variant thereof as described herein. In certain embodiments, the half-life may be at least 2, 3, 4, 5, 6, 7, 8, 9, 10, 20, 40, 60, 80, or 100 times longer than the half-life of Ab1 or a higher affinity variant thereof as described herein.

The present disclosure also contemplates an anti-TNFα antibody or antigen-binding portion with any combination of the above properties.

In some embodiments, an anti-TNFα antibody or antigen-binding portion of the present disclosure has at least one (e.g., 1, 2, 3, 4, or 5) of the following properties, in any combination:
does not form large immune complexes (i.e., two or more TNFα molecules cross-linked by three or more antibody molecules);
undergoes less degradation in vivo than Ab1 or a higher affinity variant thereof as described herein;
undergoes increased recycling to the cell surface in vivo than Ab1 or a higher affinity variant thereof as described herein;
has a longer half-life in vivo than Ab1 or a higher affinity variant thereof as described herein; and
is less immunogenic in vivo than Ab1 or a higher affinity variant thereof as described herein.

An anti-TNFα antibody or antigen-binding portion of the present disclosure can be derivatized or linked to another molecule (e.g., another peptide or protein). In general, the antibodies or portions thereof are derivatized such that TNFα binding is not affected adversely by the derivatization or labeling. Accordingly, the antibodies and antibody portions of the present disclosure are intended to include both intact and modified forms of the anti-TNFα antibodies and portions described herein. For example, an antibody or antibody portion of the present disclosure can be functionally linked (by chemical coupling, genetic fusion, noncovalent association or otherwise) to one or more other molecular entities, such as another antibody (e.g., to form a bispecific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m maleimidobenzoyl-N-hydroxysuccinimide ester) or homobifunctional (e.g., disuccinimidyl suberate). Such linkers are available, e.g., from Pierce Chemical Company, Rockford, IL.

An anti-TNFα antibody or antigen-binding portion thereof can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody or antigen-binding portion according to the present disclosure may also be labeled. As used herein, the terms “label” or “labeled” refer to incorporation of another molecule in the antibody. In some embodiments, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In some embodiments, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111 In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies of the present disclosure may be present in a neutral form (including zwitterionic forms) or as a positively or negatively-charged species. In some embodiments, the antibodies may be complexed with a counterion to form a pharmaceutically acceptable salt.

### Bispecific Binding Molecules

In a further aspect, the present disclosure provides a bispecific binding molecule having the binding specificity (e.g., comprising the antigen-binding portion, such as the six CDRs or the VH and VL) of an anti-TNFα antibody described herein and the binding specificity of a second, distinct antibody. The second antibody may be, e.g., another anti-TNFα antibody (such as another antibody described herein), or an antibody that targets a different protein, such as another cell surface molecule whose activity mediates an autoimmune or inflammatory condition. In certain embodiments, the second antibody targets IL17A, IL23, or angiopoietin 2.

The present disclosure also contemplates multispecific antibodies having the binding specificity of an anti-TNFα antibody described herein and the binding specificity of more than one additional antibody (e.g., two or three additional antibodies).

In certain embodiments, a bispecific binding molecule described herein is used in place of an anti-TNFα antibody or antigen-binding portion described herein in any aspect of the present disclosure (e.g., a therapeutic method, article of manufacture, or kit as described herein).

### Immunoconjugates

In a further aspect, the present disclosure provides an immunoconjugate comprising an anti-TNFα antibody or antigen-binding portion described herein conjugated to a therapeutic agent. In some embodiments, the therapeutic agent is an anti-inflammatory or immunosuppressive agent. In certain embodiments, the therapeutic agent is a steroid, such as a glucocorticoid receptor modulator (e.g., agonist). For example, the therapeutic agent may be selected from dexamethasone, prednisolone, budesonide, and the like. In some embodiments, the therapeutic agent may be any payload described in PCT Patent Application WO 2021/161263 or WO 2017/210471, both of which are incorporated by reference in their entirety herein.

In particular embodiments, the therapeutic agent may have the structure of Formula I below.

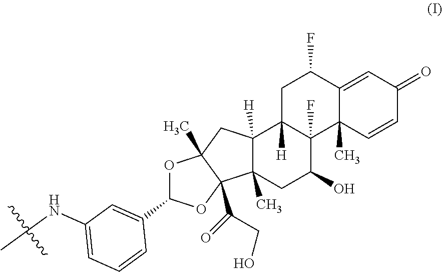

In particular embodiments, the therapeutic agent may have the structure of Formula II below.

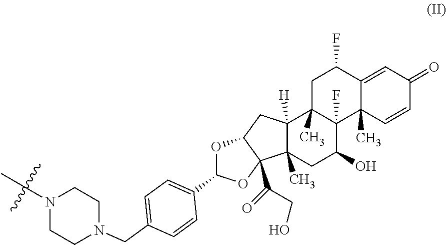

In certain embodiments, an immunoconjugate described herein is used in place of an anti-TNFα antibody or antigen-binding portion described herein in any aspect of the present disclosure (e.g., a therapeutic method, article of manufacture, or kit as described herein).

### Nucleic Acid Molecules and Vectors

The present disclosure also provides nucleic acid molecules and sequences encoding anti-TNFα antibodies or antigen-binding portions described herein. In some embodiments, different nucleic acid molecules encode the heavy chain and light chain amino acid sequences of the anti-TNFα antibody or antigen-binding portion. In other embodiments, the same nucleic acid molecule encodes the heavy chain and light chain amino acid sequences of the anti-TNFα antibody or antigen-binding portion. The present disclosure thus provides an isolated nucleic acid molecule comprising a nucleotide sequence that encodes a heavy chain or an antigen-binding portion thereof, or a nucleotide sequence that encodes a light chain or an antigen-binding portion thereof, or both, of an anti-TNFα antibody or antigen-binding portion described herein.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single- and double-stranded forms.

In any of the above embodiments, the nucleic acid molecules may be isolated. Nucleic acid molecules referred to herein as “isolated” or “purified” are nucleic acids which (1) have been separated away from the nucleic acids of the genomic DNA or cellular RNA of their source of origin; and/or (2) do not occur in nature.

In some embodiments, nucleic acid molecule(s) of the present disclosure comprise nucleotide sequences that encode H-CDR1-3 and/or L-CDR1-3 of an anti-TNFα antibody or antigen-binding portion of the present disclosure. In some embodiments, nucleic acid molecule(s) of the present disclosure comprise nucleotide sequences that encode the VH and/or VL of an anti-TNFα antibody or antigen-binding portion of the present disclosure. In some embodiments, nucleic acid molecule(s) of the present disclosure comprises nucleotide sequences that encode the HC(s) and/or LC of an anti-TNFα antibody or antigen-binding portion of the present disclosure.

In some embodiments, a nucleic acid molecule of the present disclosure comprises one or more nucleotide sequences selected from the group consisting of SEQ ID NOs: 5, 7, 9, 11, 13, 15, 29, 31, 33, 35, 37, 39, 41, 43, 45, 47, 49, 51, 53, 55, 57, 59, 61, 63, 65, 67, 69, 71, and 73.

In certain embodiments, nucleic acid molecule(s) of the present disclosure comprise the nucleotide sequences of:
SEQ ID NOs: 29 and 15;
SEQ ID NOs: 31 and 15;
SEQ ID NOs: 33 and 15;
SEQ ID NOs: 35 and 37;
SEQ ID NOs: 13 and 39;
SEQ ID NOs: 13 and 41;
SEQ ID NOs: 13 and 43;
SEQ ID NOs: 13 and 45;
SEQ ID NOs: 13 and 47;
SEQ ID NOs: 29 and 39;
SEQ ID NOs: 29 and 41;
SEQ ID NOs: 29 and 43;
SEQ ID NOs: 29 and 45;
SEQ ID NOs: 31 and 39;
SEQ ID NOs: 31 and 41;
SEQ ID NOs: 31 and 43;
SEQ ID NOs: 31 and 45;
SEQ ID NOs: 33 and 39;
SEQ ID NOs: 33 and 41;
SEQ ID NOs: 33 and 43;
SEQ ID NOs: 33 and 45;
SEQ ID NOs: 49 and 7;
SEQ ID NOs: 51 and 7;
SEQ ID NOs: 53 and 7;
SEQ ID NOs: 5 and 55;
SEQ ID NOs: 5 and 57;
SEQ ID NOs: 5 and 59;
SEQ ID NOs: 5 and 61;
SEQ ID NOs: 5 and 63;
SEQ ID NOs: 5 and 65;
SEQ ID NOs: 5 and 67;
SEQ ID NOs: 5 and 69;
SEQ ID NOs: 5 and 71; or
SEQ ID NOs: 5 and 73.

In any of the above embodiments of nucleic acid molecule(s), the nucleotide sequences may be on the same nucleic acid molecule, or on a set of nucleic acid molecules.

The present disclosure further provides a vector comprising nucleic acid molecules that encode the heavy chain(s) and light chain of an anti-TNFα antibody as described herein or an antigen-binding portion thereof. In certain embodiments, a vector of the present disclosure comprises nucleic acid molecule(s) as described herein. The vector may further comprise an expression control sequence.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

In some embodiments of the nucleic acid molecule(s) described herein, the nucleotide sequences may be arranged as two coding sequences (e.g., for a heterodimeric monovalent antibody described herein, a first coding sequence encoding the VH, VL, CH1, and Fc monomer regions, and a second coding sequence encoding a truncated HC) or three coding sequences (e.g., for a heterotrimeric monovalent antibody described herein, first and second coding sequences encoding antigen-binding protein HC and LC sequences, respectively, and a third coding sequence encoding an additional truncated HC). In certain embodiments, the coding sequences are in a polycistronic arrangement on a single nucleic acid molecule. The coding sequences of a polycistronic construct can be separated from each other, e.g., by the coding sequence of a self-cleaving peptide, or can be separated by a ribosomal internal entry site (IRES). Thus, the polycistronic construct may be transcribed as a single RNA that is processed and translated as separate polypeptides. In other embodiments, the coding sequences are on two or three separate nucleic acid molecules (e.g., for heterodimeric and heterotrimeric antibodies, respectively). The coding sequences may be under the control of the same or different promoters.

### Host Cells and Methods of Antibody Production

The present disclosure also provides methods for producing the antibodies and antigen-binding portions thereof described herein. In some embodiments, the present disclosure provides a host cell comprising nucleotide sequences that encode the heavy chain(s) and the light chain of an anti-TNFα antibody or antigen-binding portion described herein, wherein the nucleotide sequences may be on the same or different nucleic acid molecules. In some embodiments, the host cell comprises one or more vectors as described herein. In some embodiments, the present disclosure relates to a method for producing an anti-TNFα antibody or antigen-binding portion as described herein, comprising providing said host cell; culturing said host cell under conditions suitable for expression of the antibody or antigen-binding portion; and isolating the resulting antibody or antigen-binding portion. Antibodies or antigen-binding portions produced by such expression in such recombinant host cells are referred to herein as “recombinant” antibodies or antigen-binding portions. The present disclosure also provides progeny cells of such host cells, and antibodies or antigen-binding portions produced by same.

The term “recombinant host cell” (or simply “host cell”), as used herein, means a cell into which a recombinant expression vector has been introduced. By definition, a recombinant host cell does not occur in nature. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-TNFα antibodies and antigen-binding portions thereof described herein, and vectors comprising these nucleic acid molecules, can be used for transfection of a suitable mammalian, plant, bacterial or yeast host cell. In some embodiments, the nucleotide sequence encoding the light chain is transfected into the cell at a ratio of, e.g., 4:1, 2:1, or 1:1 relative to the nucleotide sequence encoding the heavy chain. In some embodiments, where the TNFα antibody has a heterotrimeric architecture as described herein, the nucleotide sequences encoding the antibody light chain, the “knob” heavy chain (e.g., the truncated heavy chain), and the “hole” heavy chain (e.g., the antibody heavy chain) may be transfected at a ratio of, e.g., 4:2:1 or 6:2:1. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

It is likely that antibodies expressed by different cell lines or in transgenic animals will have different glycosylation patterns from each other. However, all antibodies encoded by the nucleic acid molecules provided herein, or comprising the amino acid sequences provided herein are part of the present disclosure, regardless of the glycosylation state of the antibodies, and more generally, regardless of the presence or absence of post-translational modification(s).

In some embodiments, a host cell of the present disclosure comprises nucleotide sequences that encode H-CDR1-3 and/or L-CDR1-3, VH and/or VL, or HC(s) and/or LC of an anti-TNFα antibody or antigen-binding portion of the present disclosure.

In some embodiments, a host cell of the present disclosure comprises one or more nucleotide sequences selected from the group consisting of SEQ ID NOs: 5, 7, 9, 11, 13, 15, 29, 31, 33, 35, 37, 39, 41, 43, 45, 47, 49, 51, 53, 55, 57, 59, 61, 63, 65, 67, 69, 71, and 73.

In certain embodiments, a host cell of the present disclosure comprises the nucleotide sequences of:
SEQ ID NOs: 29 and 15;
SEQ ID NOs: 31 and 15;
SEQ ID NOs: 33 and 15;
SEQ ID NOs: 35 and 37;
SEQ ID NOs: 13 and 39;
SEQ ID NOs: 13 and 41;
SEQ ID NOs: 13 and 43;
SEQ ID NOs: 13 and 45;
SEQ ID NOs: 13 and 47;
SEQ ID NOs: 29 and 39;
SEQ ID NOs: 29 and 41;
SEQ ID NOs: 29 and 43;
SEQ ID NOs: 29 and 45;
SEQ ID NOs: 31 and 39;
SEQ ID NOs: 31 and 41;
SEQ ID NOs: 31 and 43;
SEQ ID NOs: 31 and 45;
SEQ ID NOs: 33 and 39;
SEQ ID NOs: 33 and 41;
SEQ ID NOs: 33 and 43;
SEQ ID NOs: 33 and 45;
SEQ ID NOs: 49 and 7;
SEQ ID NOs: 51 and 7;
SEQ ID NOs: 53 and 7;
SEQ ID NOs: 5 and 55;
SEQ ID NOs: 5 and 57;
SEQ ID NOs: 5 and 59;
SEQ ID NOs: 5 and 61;
SEQ ID NOs: 5 and 63;
SEQ ID NOs: 5 and 65;
SEQ ID NOs: 5 and 67;
SEQ ID NOs: 5 and 69;
SEQ ID NOs: 5 and 71; or
SEQ ID NOs: 5 and 73.

### Pharmaceutical Compositions

Another aspect of the present disclosure is a pharmaceutical composition comprising as an active ingredient (or as the sole active ingredient) an anti-TNFα antibody or antigen-binding portion thereof, bispecific binding molecule, or immunoconjugate of the present disclosure. In some embodiments, the pharmaceutical compositions are intended for amelioration, prevention, and/or treatment of an autoimmune or inflammatory condition, e.g., a condition described herein.

Generally, the antibodies and antigen-binding portions, bispecific binding molecules, and immunoconjugates of the present disclosure are suitable to be administered as a formulation in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present disclosure and methods for their preparation will be readily apparent to those skilled in the art. Such compositions and methods for their preparation may be found, for example, in Remington's Pharmaceutical Sciences, 19^{th} Edition (Mack Publishing Company, 1995). Pharmaceutical compositions are preferably manufactured under GMP (good manufacturing practices) conditions.

A pharmaceutical composition of the present disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Formulations of a pharmaceutical composition suitable for parenteral administration (e.g., subcutaneous administration) typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation.

### Therapeutic Uses of Antibodies and Compositions of the Present Disclosure

In some embodiments, an anti-TNFα antibody or antigen-binding portion, bispecific binding molecule, or immunoconjugate of the present disclosure is used to treat a condition in a patient, e.g., a cancer, a pulmonary condition, an intestinal condition, or a cardiac condition. In certain embodiments, the condition is an autoimmune or inflammatory condition. The patient may be a mammal, e.g., a human.

In some embodiments, the patient has a condition selected from arthritis (e.g., rheumatoid arthritis, psoriatic arthritis, gouty arthritis, juvenile idiopathic arthritis (e.g., polyarticular juvenile idiopathic arthritis), spondyloarthritis (e.g., peripheral or axial spondyloarthritis), osteoarthritis, oligoarthritis, erosive polyarthritis, or enthesitis related arthritis), Crohn's disease, ulcerative colitis, enterocolitis, inflammatory bowel disease, psoriasis (e.g., plaque psoriasis, pustular psoriasis, psoriasis vulgaris, or nail psoriasis), ankylosing spondylitis, rheymatoid spondylitis, hidradenitis suppurativa, pyoderma gangrenosum, Netherton syndrome, Dupuytren's disease, Hermansky-Pudlak syndrome, atopic dermatitis, asthma, allergy, uveitis (e.g., panuveitis or non-infectious uveitis), age-related macular degeneration, diabetic retinopathy, scleritis, Rasmussen encephalitis, asthma, sarcoidosis (e.g., cutaneous sarcoidosis), arteritis (e.g., Takayasu's arteritis or giant cell arteritis), vasculitis, Kawasaki disease, Behcet's disease, pouchitis, hepatitis, nephrotic syndrome, atherosclerosis, glomerulosclerosis (e.g., focal segmental glomerulosclerosis), multiple sclerosis, mucopolysaccharidosis (e.g., type I, II, or IV), diabetes mellitus (e.g., Type I diabetes or autoimmune diabetes), myocardial inflammation, interstitial cystitis, inflammatory bone disorder, osteomyelitis (e.g., chronic nonbacterial osteomyelitis or chronic recurrent multifocal osteomyelitis), osteoporosis, sciatica, chronic granulomatous disease, systemic lupus erythematosus (SLE), an autoimmune thyroid disorder (e.g., Hashimoto's disease), transplant rejection, graft-versus-host disease, obstructive sleep apnea, amyloidosis, a neuropsychiatric disorder (e.g., depression), and a neurodegenerative disorder (e.g., Alzheimer's disease). In some embodiments, the condition may be a chronic or acute condition, and/or may be an adult or pediatric condition.

In certain embodiments, the autoimmune or inflammatory condition is rheumatoid arthritis, psoriatic arthritis, plaque psoriasis, ankylosing spondylitis, axial spondyloarthritis, Crohn's disease, ulcerative colitis, hidradenitis suppurativa, polyarticular juvenile idiopathic arthritis, panuveitis, or Alzheimer's disease.

In some embodiments, a patient to be treated with an anti-TNFα antibody or antigen-binding portion, bispecific binding molecule, or immunoconjugate of the present disclosure has received prior treatment for the condition to be treated (e.g., autoimmune or inflammatory condition). In other embodiments, the patient has not received such prior treatment. In some embodiments, the patient has failed on a prior treatment for the condition (e.g., a prior TNFα-targeting treatment)

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

An anti-TNFα antibody or antigen-binding portion, bispecific binding molecule, or immunoconjugate of the present disclosure may be administered in a therapeutically effective amount to a patient with a condition described herein. “Therapeutically effective amount” refers to the amount of the therapeutic agent being administered that will relieve to some extent one or more of the symptoms of the disorder being treated, and/or result in clinical endpoint(s) desired by healthcare professionals.

An anti-TNFα antibody or antigen-binding portion, bispecific binding molecule, or immunoconjugate of the present disclosure may be administered without additional therapeutic treatments, i.e., as a stand-alone therapy (monotherapy). Alternatively, treatment with an anti-TNFα antibody or antigen-binding portion, bispecific binding molecule, or immunoconjugate of the present disclosure may include at least one additional therapeutic treatment (combination therapy). In some embodiments, an anti-TNFα antibody or antigen-binding portion, bispecific binding molecule, or immunoconjugate may be co-administered or formulated with another medication/drug for the treatment of the relevant condition (e.g., autoimmune or inflammatory condition).

In some embodiments, an anti-TNFα antibody or antigen-binding portion, bispecific binding molecule, or immunoconjugate of the present disclosure is administered in combination with one or more agents or treatments selected from methotrexate, prednisone, betamethasone, Enstilar®, calcipotriol, metronidazole, azathioprine, tacrolimus, hydroxychloroquine, an oral glucocorticosteroid, a non-steroidal anti-inflammatory drug (NSAID), baricitinib, ciprofloxacin, leflunomide, exenatide, teriparatide, sulfasalazine, thiopurine, 6 mercaptopurine, 2′-fucosyllactose, abatacept, etanercept, infliximab, rituximab, tocilizumab, vedolizumab, golimumab, certolizumab, ustekinumab, sarilumab, andecaliximab, anakinra, an NK cell lectin-like-receptor subfamily K antagonist (e.g., tesnatilimab), anti-thymocyte globulin, IL-2, a homocysteine modulator (e.g., vitamin B12, vitamin B6, or folic acid), and radiation.

It is understood that the antibodies and antigen-binding portions thereof, bispecific binding molecules, and immunoconjugates of the present disclosure may be used in a method of treatment as described herein, may be for use in a treatment as described herein, and/or may be for use in the manufacture of a medicament for a treatment as described herein. It is also understood that the therapies described herein may be carried out not only using the anti-TNFα antibodies or antigen-binding portions, bispecific binding molecules, or immunoconjugates thereof of the present disclosure, but also using any related pharmaceutical compositions described herein. The present disclosure also provides kits and articles of manufacture comprising the antibodies and antigen-binding portions thereof, bispecific binding molecules, immunoconjugates, or pharmaceutical compositions described herein.

### Dose and Route of Administration

The antibodies or antigen-binding portions thereof, bispecific binding molecules, and immunoconjugates of the present disclosure may be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the antibodies, bispecific binding molecules, and immunoconjugates are being administered as a stand-alone treatment or in combination with one or more additional treatments for autoimmune and/or inflammatory diseases.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the present disclosure are generally dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of the present disclosure may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens are well-known in the relevant art and would be understood to be encompassed by the skilled artisan once provided the teachings disclosed herein.

An effective amount for therapy may be measured by its ability to stabilize disease progression and/or ameliorate symptoms in a patient, and preferably to reverse disease progression. The ability of an antibody, antigen-binding portion, bispecific binding molecule, immunoconjugate, or pharmaceutical composition of the present disclosure to inhibit an autoimmune or inflammatory disease may be evaluated by in vitro assays, e.g., as described in the examples, as well as in suitable animal models that are predictive of the efficacy in humans. Suitable dosage regimens will be selected in order to provide an optimum therapeutic response in each particular situation, for example, administered as a single bolus or as a continuous infusion, and with possible adjustment of the dosage as indicated by the exigencies of each case.

The antibodies or antigen-binding portions thereof, bispecific binding molecules, immunoconjugates, and pharmaceutical compositions of the present disclosure may be administered by any method for administering peptides, proteins or antibodies accepted in the art, and are typically suitable for parenteral administration. As used herein, “parenteral administration” includes any route of administration characterized by physical breaching of a tissue of a subject and administration through the breach in the tissue, thus generally resulting in the direct administration into the blood stream, into muscle, or into an internal organ. Parenteral administration thus includes, but is not limited to, administration by injection, by application through a surgical incision, by application through a tissue-penetrating non-surgical wound, and the like. In particular, parenteral administration is contemplated to include, but is not limited to, intravenous, subcutaneous, intraperitoneal, intramuscular, intrasternal, intraarterial, intrathecal, intraurethral, intracranial, and intrasynovial injection or infusions. In a particular aspect, the antibodies or antigen-binding portions, bispecific binding molecules, immunoconjugates, or pharmaceutical compositions described herein are administered subcutaneously.

### Diagnostic Uses and Compositions

The antibodies and antigen-binding portions of the present disclosure also are useful in diagnostic processes (e.g., in vitro, ex vivo). For example, the antibodies and antigen-binding portions can be used to detect and/or measure the level of TNFα in a sample from a patient (e.g., a tissue sample, or a body fluid sample such as an inflammatory exudate, blood, serum, bowel fluid, saliva, or urine). Such detection may, for example, aid with prediction of whether or not the patient will be responsive to TNFα antibody therapy. Suitable detection and measurement methods include immunological methods such as flow cytometry, enzyme-linked immunosorbent assays (ELISA), chemiluminescence assays, radioimmunoassays, and immunohistology. The present disclosure further encompasses kits (e.g., diagnostic kits) comprising the antibodies and antigen-binding portions described herein.

### Articles of Manufacture and Kits

The present disclosure also provides articles of manufacture, e.g., kits, comprising a one or more containers (e.g., single-use or multi-use containers) containing a pharmaceutical composition of the anti-TNFα antibody or antigen-binding portion, bispecific binding molecule, or immunoconjugate of the present disclosure; optionally an additional biologically active molecule (e.g., another therapeutic agent); and instructions for use. The anti-TNFα antibody or antigen-binding portion, bispecific binding molecule, or immunoconjugate, and optional additional biologically active molecule, can be packaged separately in suitable packing such as a vial or ampoule made from non-reactive glass or plastic. In certain embodiments, the vial or ampoule holds lyophilized powder comprising the anti-TNFα antibody or antigen-binding portion, bispecific binding molecule, or immunoconjugate and/or the additional biologically active molecule. In certain embodiments, the vial or ampoule holds a concentrated stock (e.g., 2×, 5×, 10× or more) of the anti-TNFα antibody or antigen-binding portion, bispecific binding molecule, or immunoconjugate and/or the biologically active molecule. In certain embodiments, the articles of manufacture such as kits include a medical device for administering the anti-TNFα antibody or antigen-binding portion, bispecific binding molecule, or immunoconjugate and/or the biologically active molecule (e.g., a syringe and a needle); and/or an appropriate diluent (e.g., sterile water and normal saline). The articles of manufacture may further include instructions for using the anti-TNFα antibody or antigen-binding portion, bispecific binding molecule, or immunoconjugate, and optionally the additional biologically active molecule, in a method described herein. The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. Unless otherwise indicated, the recitation of a listing of elements herein includes any of the elements singly or in any combination. The recitation of an embodiment herein includes that embodiment as a single embodiment, or in combination with any other embodiment(s) herein. All publications, patents, patent applications, and other references mentioned herein are incorporated by reference in their entirety. To the extent that references incorporated by reference contradict the disclosure contained in the specification, the specification is intended to supersede and/or take precedence over any such contradictory material. Although a number of documents are cited herein, this citation does not constitute an admission that any of these documents forms part of the common general knowledge in the art.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that the present disclosure may be better understood, the following examples are set forth. These examples are for purposes of illustration only and are not to be construed as limiting the scope of the present disclosure in any manner.

### EXAMPLES

### Example 1. Design and Expression of Ab1 Variants

To enable the rapid identification of higher affinity Ab1 variants with pH sensitive binding we synthesized and expressed Fabs in bacteria using a phage expression system, using previously described higher affinity variants of Ab1 as a starting point. Subsequently, pH switch variants of higher affinity Ab1 variants A1 and 4.2a-6 were expressed and characterized.

### Materials and Methods

Cloning of Fab Variants into Phage Expression System

DNA encoding the heavy and light chain variable regions of all constructs was synthesized as gBlocks (Integrated DNA Technologies) and was cloned into a phage expression vector that contained human kappa light chain constant domain and human G1 heavy chain constant domain 1. In addition, the vector contained a his-tag and hemagglutinin A tag at the carboxy-terminal end of the heavy chain to facilitate purification and detection.

Expression and Quantitation of Fab in the Periplasmic Space of E. coli.

Cloning was verified by expressing and quantitating Fab in the periplasmic space of E. coli. Briefly, XL-0 bacteria were grown in 2× YT medium at 37° C. until the culture reached a density of 0.9-1.1 at OD600. Isopropyl □-D-thiogalactoside was then added to the cells to a final concentration of 1 mM and 4.0 mL of culture was transferred to a 14 mL snap-top tube. Each tube was transfected with 4 μL of high titer phage stock and the cultures were placed in a shaker (225 rpm) at 37° C. One hour later, the temperature was shifted to 25° C. and the cultures were grown for an additional 14-16 h. The cells were collected by centrifugation at 3900 rpm for 30 min in an Eppendorf 5810R centrifuge (˜3,200×g), the supernatant was decanted, and the cells were resuspended in 0.25 mL of lysis buffer (30 mM Tris, pH 8.0, 2 mM EDTA, 20% sucrose, 2 mg/ml lysozyme, 5 U/mL Dnase I) and placed on ice for 30 min. The cell suspension was transferred to a 1.5 mL tube and cell debris was pelleted by centrifugation at 15,000 rpm for 15 min in an Eppendorf 5424 microfuge (˜21,000×g). The supernatant was removed carefully without disturbing the pellet and was stored at 4° C. until use.

In order to quantitate Fab expression, a 96-well Costar-3366 plate was coated with 50 μL/well of 2 μg/mL sheep anti-human Fd (Southern Biotech, Prod. #2046-01) in PBS overnight at 4° C. The plate was washed three times with PBS containing 0.05% Tween 20 (PBS-T) and 50 μL/well of sample dilutions was added. Sample dilutions were performed with 1% BSA-PBS. A standard curve was generated using human Fab (Rockland, Prod. #009-01015) diluted serially 3-fold, beginning at 1 μg/mL. The plates were incubated 1 h at 25° C., washed three times with PBS-T, and incubated with 50 μL/well of anti-kappa HRP conjugate (Southern Biotech, Prod. #2060-05), diluted 5,000-fold in PBS-T, for 1 h at 25° C. The plate was washed three times with PBS-T, then developed with 50 μL/well 1-Step Ultra TMB-ELISA (ThermoFisher Scientific, Prod. #34028). The reaction was terminated by the addition of 2 N H₂SO₄ and the A450 was determined before and after addition of H₂SO₄, respectively, using a Spectramax plate reader.

### Results

The variants expressed and characterized are summarized in Table 1 below.

TABLE 1
Variable Domain Sequences of Ab1 and Higher Affinity Variants

| | | SEQ ID NO | | | |
|---|---|---|---|---|---|
| | | Heavy Chain | | Light Chain | |
| | pH Switch | Variable (VH) | | Variable (VL) | |
| Construct | Template | DNA | Protein | DNA | Protein |
| Ab1 | — | 1 | 2 | 3 | 4 |
| A1 | — | 5 | 6 | 7 | 8 |
| cb1-3 | — | 9 | 10 | 11 | 12 |
| 4.2a-6 | — | 13 | 14 | 15 | 16 |
| Ab4 | — | 17 | 18 | 19 | 20 |
| AF-M2631 | Ab1 | 21 | 22 | 3 | 4 |
| AF-M2632 | Ab1 | 23 | 24 | 3 | 4 |
| AF-M2635 | Ab1 | 1 | 2 | 25 | 26 |
| AF-M2637 | Ab1 | 1 | 2 | 27 | 28 |
| 4.2a-6-VH1 | 4.2a-6 | 29 | 30 | 15 | 16 |
| 4.2a-6-VH2 | 4.2a-6 | 31 | 32 | 15 | 16 |
| 4.2a-6-VH3 | 4.2a-6 | 33 | 34 | 15 | 16 |
| 4.2a-6-H | 4.2a-6 | 35 | 36 | 37 | 38 |
| 4.2a-6-VL1 | 4.2a-6 | 13 | 14 | 39 | 40 |
| 4.2a-6-VL2 | 4.2a-6 | 13 | 14 | 41 | 42 |
| 4.2a-6-VL4 | 4.2a-6 | 13 | 14 | 43 | 44 |
| 4.2a-6-VL5 | 4.2a-6 | 13 | 14 | 45 | 46 |
| 4.2a-6-VL7 | 4.2a-6 | 13 | 14 | 47 | 48 |
| 4.2a-6- | 4.2a-6 | 29 | 30 | 39 | 40 |
| VH1 × VL1 | | | | | |
| 4.2a-6- | 4.2a-6 | 29 | 30 | 41 | 42 |
| VH1 × VL2 | | | | | |
| 4.2a-6- | 4.2a-6 | 29 | 30 | 43 | 44 |
| VH1 × VL4 | | | | | |
| 4.2a-6- | 4.2a-6 | 29 | 30 | 45 | 46 |
| VH1 × VL5 | | | | | |
| 4.2a-6- | 4.2a-6 | 31 | 32 | 39 | 40 |
| VH2 × VL1 | | | | | |
| 4.2a-6- | 4.2a-6 | 31 | 32 | 41 | 42 |
| VH2 × VL2 | | | | | |
| 4.2a-6- | 4.2a-6 | 31 | 32 | 43 | 44 |
| VH2 × VL4 | | | | | |
| 4.2a-6- | 4.2a-6 | 31 | 32 | 45 | 46 |
| VH2 × VL5 | | | | | |
| 4.2a-6- | 4.2a-6 | 33 | 34 | 39 | 40 |
| VH3 × VL1 | | | | | |
| 4.2a-6- | 4.2a-6 | 33 | 34 | 41 | 42 |
| VH3 × VL2 | | | | | |
| 4.2a-6- | 4.2a-6 | 33 | 34 | 43 | 44 |
| VH3 × VL4 | | | | | |
| 4.2a-6- | 4.2a-6 | 33 | 34 | 45 | 46 |
| VH3 × VL5 | | | | | |
| A1 VH1 | A1 | 49 | 50 | 7 | 8 |
| A1 VH2 | A1 | 51 | 52 | 7 | 8 |
| A1 VH3 | A1 | 53 | 54 | 7 | 8 |
| A1 VL1 | A1 | 5 | 6 | 55 | 56 |
| A1 VL2 | A1 | 5 | 6 | 57 | 58 |
| A1 VL3 | A1 | 5 | 6 | 59 | 60 |
| A1 VL4 | A1 | 5 | 6 | 61 | 62 |
| A1 VL5 | A1 | 5 | 6 | 63 | 64 |
| A1 VL6 | A1 | 5 | 6 | 65 | 66 |
| A1 VL7 | A1 | 5 | 6 | 67 | 68 |
| A1 L Q27H | A1 | 5 | 6 | 69 | 70 |
| A1 L G28H | A1 | 5 | 6 | 71 | 72 |
| A1 L I297H | A1 | 5 | 6 | 73 | 74 |

Table 2 below lists the CDR sequences for the variants.

TABLE 2
CDR Sequences of Ab1 and Select Higher Affinity Variants

| Construct | H-CDR1 | H-CDR2 | H-CDR3 | L-CDR1 | L-CDR2 | L-CDR3 |
|---|---|---|---|---|---|---|
| Ab1 | 75 | 76 | 77 | 78 | 79 | 80 |
| A1 | 81 | 76 | 82 | 83 | 79 | 80 |
| cb1-3 | 81 | 76 | 77 | 84 | 85 | 86 |
| 4.2a-6 | 87 | 76 | 77 | 88 | 89 | 90 |
| 4.2a-6-VH1 | 87 | 76 | 97 | 88 | 89 | 90 |
| 4.2a-6-VH2 | 87 | 76 | 98 | 88 | 89 | 90 |
| 4.2a-6-VH3 | 87 | 76 | 101 | 88 | 89 | 90 |
| 4.2a-6-H | 87 | 76 | 102 | 88 | 89 | 103 |
| 4.2a-6-VL1 | 87 | 76 | 77 | 104 | 89 | 90 |
| 4.2a-6-VL2 | 87 | 76 | 77 | 88 | 89 | 105 |
| 4.2a-6-VL4 | 87 | 76 | 77 | 88 | 89 | 106 |
| 4.2a-6-VL5 | 87 | 76 | 77 | 107 | 89 | 90 |
| 4.2a-6-VL7 | 87 | 76 | 77 | 104 | 89 | 108 |
| 4.2a-6- | 87 | 76 | 97 | 104 | 89 | 90 |
| VH1 × VL1 | | | | | | |
| 4.2a-6- | 87 | 76 | 97 | 88 | 89 | 105 |
| VH1 × VL2 | | | | | | |
| 4.2a-6- | 87 | 76 | 97 | 88 | 89 | 106 |
| VH1 × VL4 | | | | | | |
| 4.2a-6- | 87 | 76 | 97 | 107 | 89 | 90 |
| VH1 × VL5 | | | | | | |
| 4.2a-6- | 87 | 76 | 98 | 104 | 89 | 90 |
| VH2 × VL1 | | | | | | |
| 4.2a-6- | 87 | 76 | 98 | 88 | 89 | 105 |
| VH2 × VL2 | | | | | | |
| 4.2a-6- | 87 | 76 | 98 | 88 | 89 | 106 |
| VH2 × VL4 | | | | | | |
| 4.2a-6- | 87 | 76 | 98 | 107 | 89 | 90 |
| VH2 × VL5 | | | | | | |
| 4.2a-6- | 87 | 76 | 101 | 104 | 89 | 90 |
| VH3 × VL1 | | | | | | |
| 4.2a-6- | 87 | 76 | 101 | 88 | 89 | 105 |
| VH3 × VL2 | | | | | | |
| 4.2a-6- | 87 | 76 | 101 | 88 | 89 | 106 |
| VH3 × VL4 | | | | | | |
| 4.2a-6- | 87 | 76 | 101 | 107 | 89 | 90 |
| VH3 × VL5 | | | | | | |
| A1 VH1 | 81 | 76 | 109 | 83 | 79 | 80 |
| A1 VH2 | 81 | 76 | 110 | 83 | 79 | 80 |
| A1 VH3 | 81 | 76 | 111 | 83 | 79 | 80 |
| A1 VL1 | 81 | 76 | 82 | 112 | 79 | 80 |
| A1 VL2 | 81 | 76 | 82 | 83 | 79 | 99 |
| A1 VL3 | 81 | 76 | 82 | 83 | 79 | 113 |
| A1 VL4 | 81 | 76 | 82 | 83 | 79 | 100 |
| A1 VL5 | 81 | 76 | 82 | 114 | 79 | 80 |
| A1 VL6 | 81 | 76 | 82 | 83 | 79 | 115 |
| A1 VL7 | 81 | 76 | 82 | 112 | 79 | 116 |
| A1 L Q27H | 81 | 76 | 82 | 117 | 79 | 80 |
| A1 L G28H | 81 | 76 | 82 | 118 | 79 | 80 |
| A1 L I297H | 81 | 76 | 82 | 119 | 79 | 80 |

### Example 2. Characterization of Ab1 Fab, Ab1 Variant Fabs, and Ab4 Fab by ELISA

Introduction of histidine residues into CDRs to identify variants with pH-sensitive binding often results in diminished binding at pH 7.4. Consequently, to generate Ab1 pH switch variants with higher affinity at pH 7.4, we used previously identified higher affinity Ab1 variants (Table 1: A1, cb1-3, and 4.2a-6) as a starting point (template) for introducing histidine residues into CDRs. Prior to generating the pH switch variants, the relative binding of Ab1 Fab and Ab1 variant Fabs was characterized by ELISA. Additionally, the relative binding of Ab4 Fab, which has been reported to bind TNFα with higher affinity than Ab1, was characterized.

### Materials and Methods

ELISA Characterization of Fab Binding to Soluble Biotinylated TNFα with Long Wash in the Presence of Soluble TNFα.

A 96-well Costar-3366 plate was coated with 50 μL/well of 2 μg/mL sheep anti-human Fd (Southern Biotech, Prod. #2046-01) in PBS for 1 hour at room temperature. The plate was washed four times with PBS containing 0.05% Tween 20 (PBS-T) and blocked with 100 μL/well with 1% BSA-PBS for 1 hour at room temperature. Block was removed and 50 μL/well of 1 μg/mL sample was added. Sample dilutions were performed with 1% BSA-PBS. The plate was washed four times with PBS containing 0.05% Tween 20 (PBS-T), then biotinylated human TNFα was serially diluted 3-fold starting at 60 nM in B-PBS and incubated for 1 h at 25° C. (50 μL/well). The plate was washed four times with PBS-T and 50 μL/well of 100 nM human TNFα in 1% BSA was added for 20 h at 25° C. The plates were washed four times with PBS-T and incubated with 50 μL/well of Neutravidin HRP (ThermoFisher Scientific, cat. #31030), diluted 2,000-fold in B-PBS for 1 h at 25° C. The plate was washed four times with PBS-T, then developed with 50 μL/well 1-Step Ultra TMB-ELISA (ThermoFisher Scientific, Prod. #34028). The reaction was terminated by the addition of 2 N H₂SO₄ and the A450 was determined before and after addition of H₂SO₄, respectively, using a Spectramax plate reader.

### Results

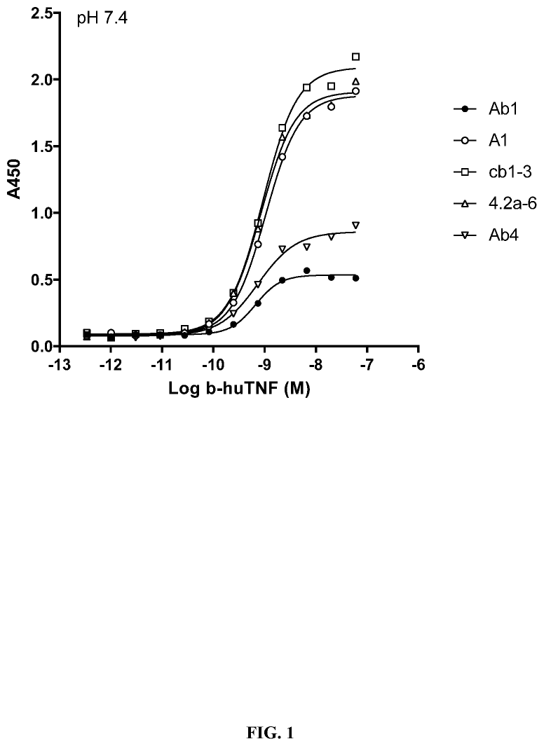

Characterization of variants using typical ELISA conditions with multiple rapid washes did not enable the binding affinities of the Fab variants to be distinguished from Ab1 Fab (data not shown). However, binding of soluble, biotinylated TNFα to immobilized Fab followed by a prolonged dissociation step (20 h) in the presence of 100 nM non-biotinylated TNFα, enabled binding affinity differences to be distinguished. Under these conditions, Ab1 Fab binding was weakest, as evidenced by the lowest maximal binding signal (FIG. 1). Ab4 Fab and the Ab1 Fab variants all bound TNFα with higher affinity than Ab1 Fab (FIG. 1). Ab4 Fab bound more tightly than Ab1 Fab, but weaker than the three affinity-enhanced Ab1 Fab variants, A1, cb1-3, and 4.2a-6 (FIG. 1). Consequently, the variable regions of A1, cb1-3 and 4.2a-6 could all serve as templates for creating higher affinity Ab1 pH switch variants.

### Example 3. Characterization of pH Sensitivity of Binding of Ab1, Ab1 Variant, Ab4, AF-M2631 and AF-M2637 Fabs

Next, the pH sensitivity of binding of the higher affinity Ab1 Fab variants was compared to Fabs of Ab1, Ab4, and previously identified pH switch variants of Ab1, AF-M2631 and AF-M2637. A modified ELISA was used to enable discernment of the relative binding strengths of the TNFα Fabs at both pH 7.4 and pH 6.0. Briefly, following Fab binding to immobilized TNFα□ a prolonged dissociation step (2 h) in the presence of 100 nM TNFα at either pH 7.4 or pH 6.0 was performed.

### Materials and Methods

ELISA Characterization of Fab Binding to Immobilized TNFα with Long Wash in the Presence of Soluble TNFα

A 96-well Costar-3366 plate was coated with 50 μL/well of 1 μg/mL human TNFα (Genscript cat. #Z01001) in PBS for one hour at room temperature. The plate was rinsed twice with PBS-T and blocked with 100 μL/well of 1% BSA in PBS (B-PBS) for 1 h at 25° C. Fab samples were serially diluted 3-fold starting at 40 nM in B-PBS and were incubated for 1 h at 25° C. (50 μL/well). The plate was washed four times with PBS-T and 50 L/well of 100 nM human TNFα in 1% BSA was added for 2 h at 25° C. The plate was washed four times with PBS-T and 50 μL/well of anti-human kappa, HRP conjugate (Southern Biotech, Prod. #2060-05) diluted 5,000-fold in B-PBS was added for 1 h at 25° C. The plate was washed three times with PBS-T, then developed with 50 μL/well 1-Step Ultra TMB-ELISA (ThermoFisher Scientific, Prod. #34028). The reaction was terminated by the addition of 2 N H₂SO₄ and the A450 was determined before and after addition of H₂SO₄, respectively, using a Spectramax plate reader.

### Results

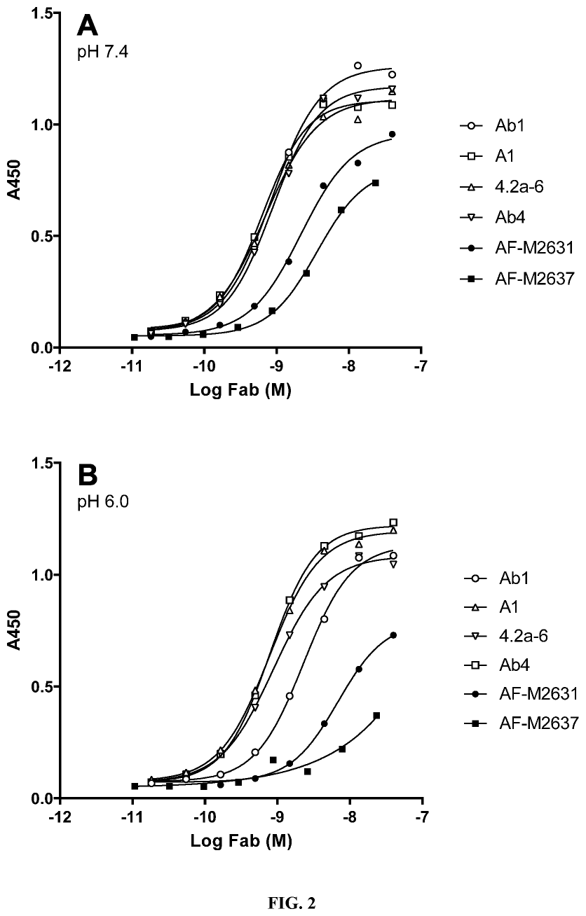

Characterization of the binding of the Fabs to immobilized TNFα at pH 7.4 demonstrated that Ab1, A1, 4.2a-6 and Ab4 all bound with similar high affinities (FIG. 2A, open symbols). The Ab1 pH switch variant Fabs AF-M2631 and AF-M2637 bound with lower affinities, with AF-M2631 binding more tightly than AF-M2637 (FIG. 2A, closed circles and closed squares, respectively). Following a prolonged pH 6.0 dissociation step, the binding of pH switch variant Fabs AF-M2631 and AF-M2637 was significantly diminished relative to the other variants (FIG. 2B, compare closed symbols versus open symbols). Additionally, Ab1 Fab appeared to display pH-sensitive binding to a greater extent than was observed for the higher affinity Ab1 Fab variants A1 and 4.2a-6 or Ab4 Fab (FIG. 2B, compare open circles versus other open symbols). These data demonstrate that the variable regions of A1 and 4.2a-6 Fabs may serve as templates for engineering pH switch variants that bind more tightly than Ab1-based pH switch variants at pH 7.4.

### Example 4. Expression and Characterization of Heavy Chain or Light Chain pH Switch Variant Fabs Using 4.2a-6 as a Template

Next, heavy chain and light chain variants of 4.2a-6 containing certain histidine mutations in CDRs were expressed as Fabs in bacteria and binding following prolonged dissociation at pH 7.4 or pH 6.0 was characterized. Ab1, AF-M2631, AF-M2637 and eight 4.2a-6 variants, termed 4.2a-6-VH1, 4.2a-6-VH2, 4.2a-6-VH3, 4.2a-6-H, 4.2a-6-VL1, 4.2a-6-VL2, 4.2a-6-VL4, and 4.2a-6-VL5 (Table 1) were analyzed. The ELISA method used to characterize these variants was described in Example 3.

### Results

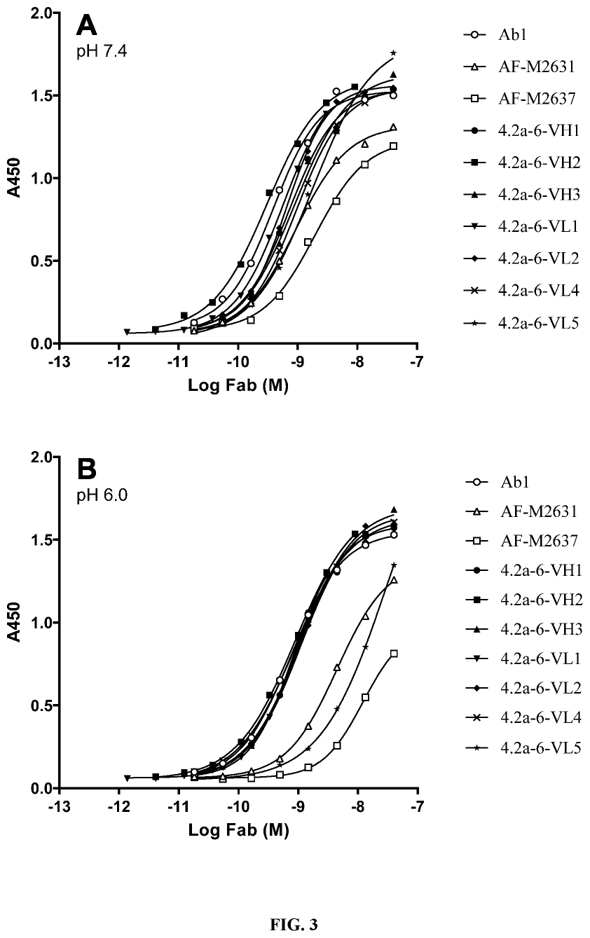

All histidine heavy chain or light chain CDR Fab variants of the 4.2a-6 template bound TNFα better than AF-M2631 Fab following a prolonged pH 7.4 dissociation step (FIG. 3A, compare closed symbols with open triangles). Most variants displayed similar, though somewhat weaker, binding strength than Ab1 Fab (FIG. 3A, compare closed symbols with open circles). Fab variant 4.2a-6-VH2 displayed stronger binding than Ab1 Fab (FIG. 3A, compare closed squares with open circles) while Fab variant 4.2a-6-VL1 displayed very similar binding to Ab1 Fab (FIG. 3A, compare closed inverted triangles with open circles). The majority of the Fab variants bound similarly to Ab Fab following a prolonged pH 6.0 dissociation step (FIG. 3B, compare closed symbols with open circles). However, Fab variant 4.2a-6-VL5 displayed binding that was diminished more than AF-M2631 Fab (FIG. 3B, compare closed stars with open triangles). Fab variant 4.2a-6-VL5 displayed robust pH dependent binding, displaying strong binding at pH 7.4 and significantly diminished binding at pH 6.0. Based on sequence homology, Fab variant 4.2a-6-VL7 is expected to display similar characteristics to Fab variant 4.2a-6-VL5.

### Example 5. Expression and Characterization of Heavy Chain and Light Chain Combinatorial pH Switch Variant Fabs Using 4.2a-6 as a Template

The individual 4.2a-6 heavy chain and light chain pH switch variant sequences were combined (Table 1, 4.2a-6 template combinatorial pH switch variants) to determine if more potent pH sensitivity at pH 6.0 could be identified. The ELISA method used to characterize these variants was described in Example 3.

### Results

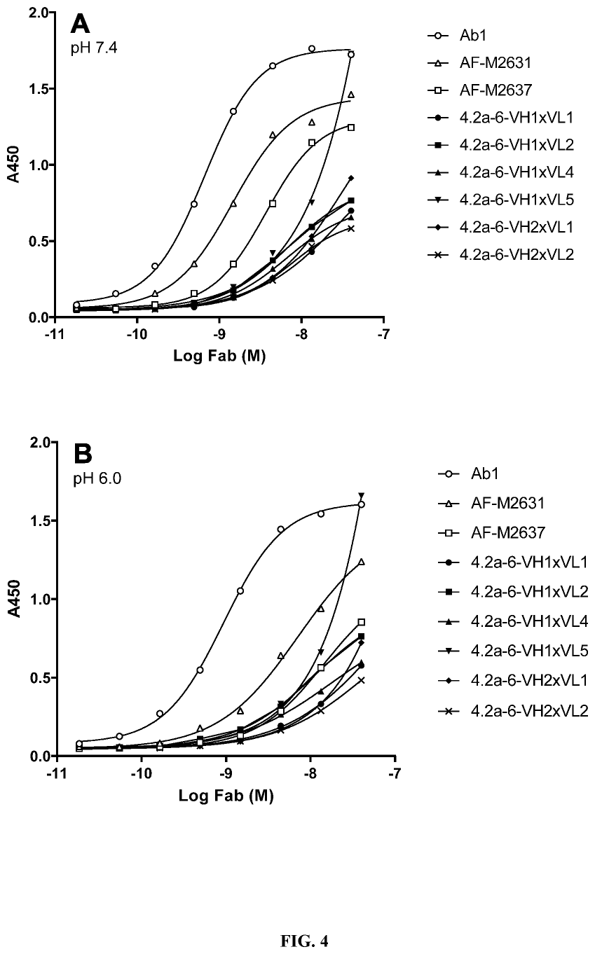

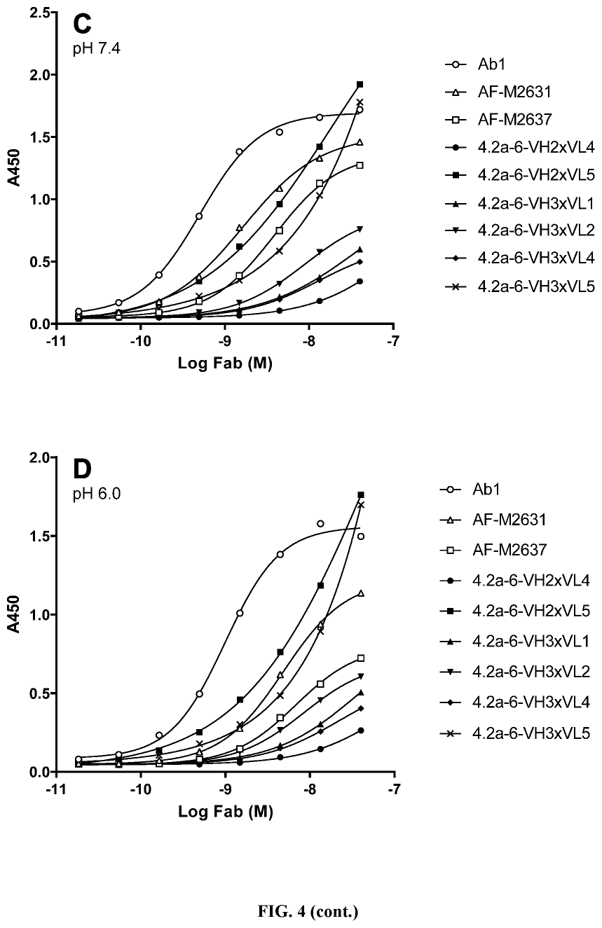

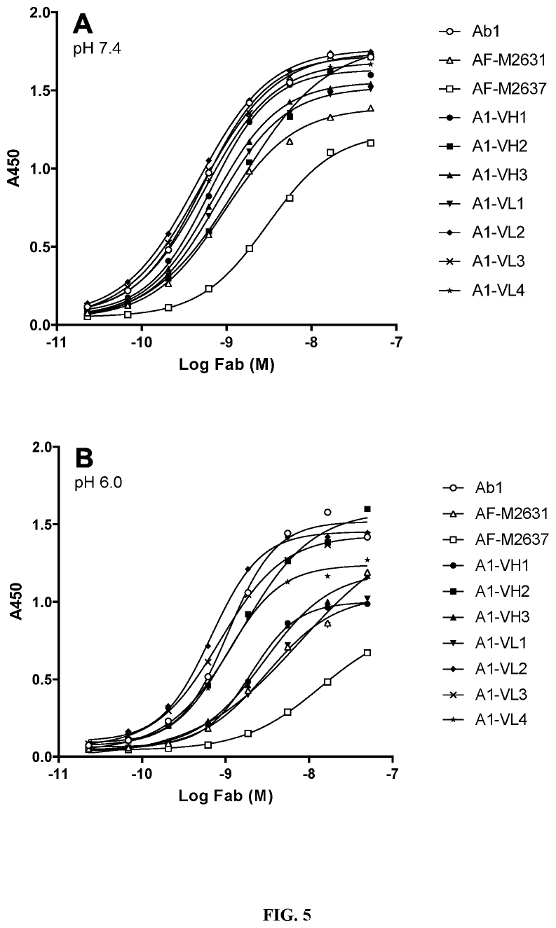

All of the combinatorial Fab variants containing the 4.2a-6-VH1 heavy chain displayed significantly weaker binding than AF-M2637 Fab following prolonged dissociation at pH 7.4 (FIG. 4A, compare closed symbols with open symbols). Moreover, following a prolonged dissociation at pH 6.0 the variants did not display significantly diminished binding, indicating the 4.2a-6-VH1-containing combinatorial histidine mutations were not effective for creating a pH switch upon the 4.2a-6 template (FIG. 4B). Similar results were observed with the combinatorial Fab variants containing the 4.2a-6-VH2 heavy chain. Specifically, 4.2a-6-VH2×VL1 and 4.2a-6-VH2×VL2 displayed weak binding following prolonged dissociation at pH 7.4 (FIG. 4A) and little pH switch activity at pH 6.0 (FIG. 4B). Variant 4.2a-6-VH2×VL4 displayed little binding following prolonged dissociation at both pH 7.4 (FIG. 4C) and pH 6.0 (FIG. 4D). Variant 4.2a-6-VH2×VL5 displayed greater binding than AF-M2637, but less than AF-M2631, following prolonged dissociation at pH 7.4 (FIG. 4C). However, 4.2a-6-VH2×VL5 did not display a pH switch during dissociation at pH 6.0 (FIG. 4D). All combinatorial Fab variants containing the 4.2a-6-VH3 heavy chain displayed significantly weaker binding than AF-M2637 following prolonged dissociation at pH 7.4 (FIG. 4C, compare closed symbols with open symbols). Moreover, following a prolonged dissociation at pH 6.0 the variants did not display significantly diminished binding, indicating the 4.2a-6-VH3-containing combinatorial histidine mutations were not effective for creating a pH switch upon the 4.2a-6 template (FIG. 4D). Collectively, these data indicate that the combinatorial pH switches built upon the 4.2a-6 template in this study are not useful in a monovalent format.

### Example 6. Expression and Characterization of Heavy Chain or Light Chain pH Switch Variant Fabs Using A1 as a Template

Next, heavy chain and light chain variants of A1 containing certain histidine mutations in CDRs were expressed as Fabs in bacteria and binding following prolonged dissociation at pH 7.4 or pH 6.0 was characterized. Ab1, AF-M2631, AF-M2637 and thirteen A1 variants, termed A1-VH1, A1-VH2, A1-VH3, A1-VL1, A1-VL2, A1-VL3, A1-VL4, A1-VL5, A1-VL-6, A1-VL7, A1 LC Q27H, A1 LC G28H, and A1 LC 129H, were analyzed (Table 1). The ELISA method used to characterize these variants was described in Example 3.

### Results

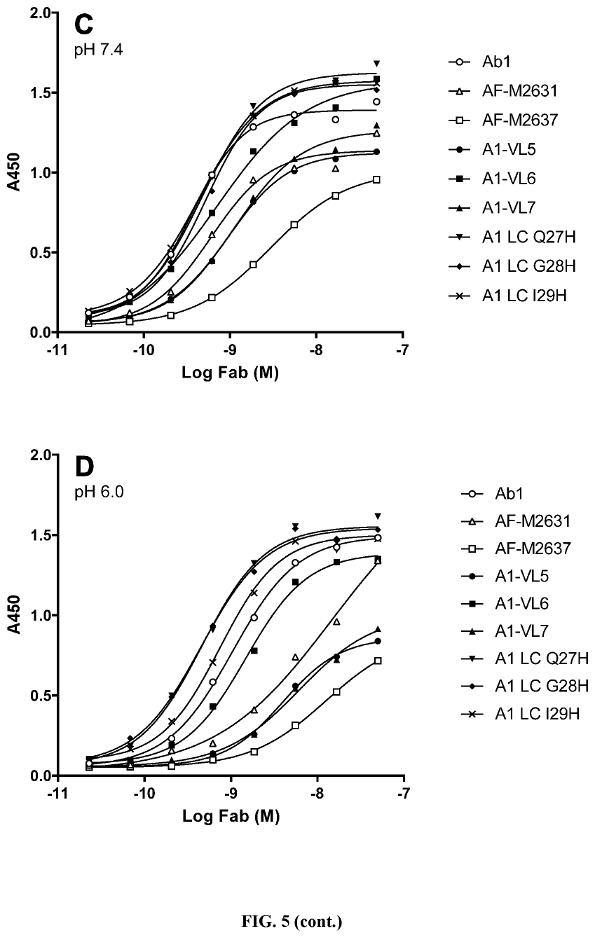

All Fab variants, except A1-VL5 and A1-VL7, bound more tightly than AF-M2631 Fab following a prolonged dissociation at pH 7.4 (FIG. 5A and FIG. 5C). A1-VL5 and A1-VL7 displayed binding similar to AF-M2631 Fab (FIG. 5C compare closed triangles and closed circles to open triangles). Fab variants A1-VH1 (FIG. 5B, closed circles), A1-VH3 (FIG. 5B, closed triangles), A1-VL1 (FIG. 5B, inverted closed triangles), A1-VL5 (FIG. 5D, closed circles), and A1-VL7 (FIG. 5D, closed triangles) all displayed enhanced dissociation at pH 6.0 relative to pH 7.4. Consequently, all of these monovalent Fab variants are potentially useful pH sensitive modulators of TNFα binding and neutralization. In contrast, Fab variants A1 LC Q27H, A1 LC G28H, and A1 LC 129H bound more tightly than Ab1 Fab following prolonged dissociation at pH 6.0. Therefore, these three Fab variants are not useful as pH switches.

### Example 7. Characterization of Certain A1 Template Heavy Chain or Light Chain pH Switch Fab Variants

Certain Fab variants identified in Example 6 were re-characterized and compared to the Fabs of Ab1, AF-M2631, AF-M2637 and A1. The ELISA method used to characterize these variants was described in Example 3.

### Results

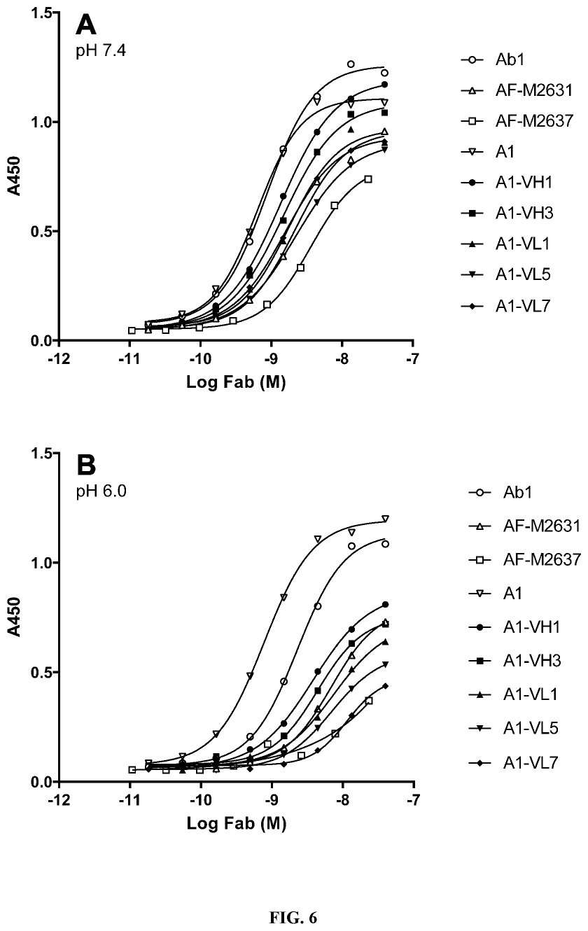

All Fab variants displayed stronger binding than AF-M2637 Fab following prolonged dissociation at pH 7.4 (FIG. 6A, compare closed symbols with open squares). Variants A1-VH1 (closed circles) and A1-VH3 (closed squares) bound more tightly than AF-M2631 Fab (open triangles), while A1-VL1 (closed triangles), A1-VL5 (closed inverted triangles) and A1-VL7 (closed diamonds) displayed similar binding to AF-M2631 Fab. Template A1 Fab (open inverted triangles) binding was indistinguishable from Ab1 Fab (open circles) under these assay conditions. All variants displayed enhanced dissociation at pH 6.0 (FIG. 6B, closed symbols). A1-VH1 (closed circles) and A1-VH3 (closed squares) displayed slightly tighter binding than AF-M2631 (open triangles) at pH 6.0 dissociation, while A1-VL1 (closed triangles) and A1-VL5 (closed inverted triangles) weaker binding than AF-M2631 at pH 6.0 dissociation. A1-VL7 displayed the weakest binding at pH 6.0 dissociation, comparable to AF-M2637 (open squares). All variants displayed robust pH dependent binding in the monovalent Fab format, displaying strong binding at pH 7.4 and substantially reduced binding at pH 6.0.

### Example 8. Characterization of Fab Variant Binding to Murine TNFα

The effect of diminished binding of the variants at pH 6.0 on immunogenicity can be screened using wild-type mice. However, prior to performing in vivo studies, the binding of the variants to murine TNFα was characterized by ELISA to determine if the pH switch activity observed with human TNFα was preserved with murine TNFα.

### Materials and Methods

ELISA Characterization of Fab Binding to Soluble Biotinylated Murine TNFα with Long Wash in the Presence of Soluble TNFα

A 96-well Costar-3366 plate was coated with 50 μL/well of 2 μg/mL sheep anti-human Fd (Southern Biotech, Prod. #2046-01) in PBS for 1 hour at room temperature. The plate was washed four times with PBS containing 0.05% Tween 20 (PBS-T) and blocked with 100 μL/well of 1% BSA-PBS for 1 hour at room temperature. Block was removed and 50 μL/well of 0.5 ug/mL sample was added. Sample dilutions were performed with 1% BSA-PBS. The plate was washed four times with PBS containing 0.05% Tween 20 (PBS-T), then biotinylated murine (Acro biosystems) TNFα was serially diluted 3-fold starting at 30 nM in B-PBS and incubated for 1 h at 25° C. (50 μL/well). The plates were washed in 500 mL of PBS-T, pH 6.0 or PBS-T, pH 7.4 for 1 hour. PBS-T was removed from the plates every 10 minutes during this wash. The plates were then washed four times with PBS-T, and incubated with 50 μL/well of Neutravidin HRP (ThermoFisher Scientific, cat. #31030), diluted 2,000-fold in B-PBS for 1 h at 25° C. The plate was washed four times with PBS-T, then developed with 50 μL/well 1-Step Ultra TMB-ELISA (ThermoFisher Scientific, Prod. #34028). The reaction was terminated by the addition of 2 N H₂SO₄ and the A450 was determined before and after addition of H₂SO₄, respectively, using a Spectramax plate reader.

### Results

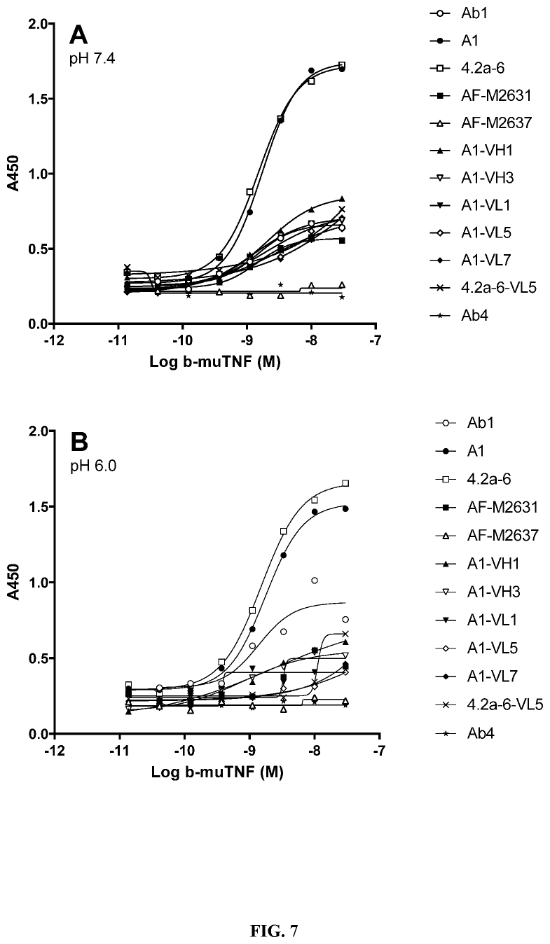

All A1 template pH switches identified in Example 7 and the 4.2a-6 template pH switch, 4.2a-6-VL5 identified in Example 4, bound murine TNFα at pH 7.4 (FIG. 7A). A1 (closed circles) and 4.2a-6 (open squares) bound murine TNFα tightly, while Ab4 and control AF-M2637 did not bind to murine TNFα under the conditions used for this assay (stars and open triangles, respectively). Ab1, control AF-M2631, and all other variants displayed similar, intermediate binding activity.

The binding of A1 (closed circles) and 4.2a-6 (open squares) to murine TNFα following pH 6.0 dissociation remained strong, indicating that the binding was not pH sensitive (FIG. 7B). Similarly, Ab1 (open circles) binding was only slightly diminished following pH 6.0 dissociation. The binding of all variants, with the exception of AF-M2631 (closed squares), A1-VH1 (closed triangles) and A1-VH3 (open, inverted triangles) was significantly reduced following pH 6.0 dissociation. Consequently, all variants are suitable for testing in mice, though certain ones (AF-M2631, A1-VH1 and A1-VH3) may display sub-optimal pH sensitivity.

What is claimed is:

1. An anti-TNFα antibody or an antigen-binding portion thereof that binds to the same epitope of human TNFα as a reference antibody comprising:
a) a heavy chain variable domain (VH) that comprises the amino acid sequence of SEQ ID NO: 6 and a light chain variable domain (VL) that comprises the amino acid sequence of SEQ ID NO: 8;
b) a VH that comprises the amino acid sequence of SEQ ID NO: 10 and a VL that comprises the amino acid sequence of SEQ ID NO: 12; or
c) a VH that comprises the amino acid sequence of SEQ ID NO: 14 and a VL that comprises the amino acid sequence of SEQ ID NO: 16;
wherein said anti-TNFα antibody or antigen-binding portion comprises VH and VL at least 90% identical to the VH and VL of the reference antibody, respectively; and
wherein said anti-TNFα antibody or antigen-binding portion has a binding affinity for TNFα that is lower at pH 6.0 than at pH 7.4, and optionally is monovalent.

2. The anti-TNFα antibody of claim 1, wherein said antibody comprises
a) a monovalent antigen-binding protein comprising a heavy chain (HC) that comprises a VH at least 90% identical to the VH of the reference antibody and a light chain (LC) that comprises a VL at least 90% identical to the VL of the reference antibody; and
b) a truncated HC lacking the variable domain and CH1 domain;
wherein the antigen-binding protein HC and the truncated HC are capable of dimerization.

3. The anti-TNFα antibody of claim 2, wherein the antigen-binding protein HC and the truncated HC comprise knobs-into-holes modifications, optionally wherein the antigen-binding protein HC is of isotype subclass IgG1 and comprises mutations T366S, L368A, and Y407A in the CH3 domain, and the truncated HC is of isotype subclass IgG1 and comprises the mutation T366W in the CH3 domain, wherein the residues are numbered according to the Eu system.

4. The anti-TNFα antibody of claim 2 or 3, wherein the antigen-binding protein HC is of isotype subclass IgG1 and comprises the mutation Y349C, and the truncated HC is of isotype subclass IgG1 and comprises the mutation S354C, wherein the residues are numbered according to the Eu system.

5. An anti-TNFα antibody or antigen-binding portion thereof that comprises heavy chain (HC) CDR1-3 and light chain (LC) CDR1-3 comprising:
a) SEQ ID NOs: 87, 76, 97, 88, 89, 90, respectively;
b) SEQ ID NOs: 87, 76, 98, 88, 89, 90, respectively;
c) SEQ ID NOs: 87, 76, 101, 88, 89, 90, respectively;
d) SEQ ID NOs: 87, 76, 102, 88, 89, 103, respectively;
e) SEQ ID NOs: 87, 76, 77, 104, 89, 90, respectively;
f) SEQ ID NOs: 87, 76, 77, 88, 89, 105, respectively;
g) SEQ ID NOs: 87, 76, 77, 88, 89, 106, respectively;
h) SEQ ID NOs: 87, 76, 77, 107, 89, 90, respectively;
i) SEQ ID NOs: 87, 76, 77, 104, 89, 108, respectively;
j) SEQ ID NOs: 87, 76, 97, 104, 89, and 90, respectively;
k) SEQ ID NOs: 87, 76, 97, 88, 89, and 105, respectively;
l) SEQ ID NOs: 87, 76, 97, 88, 89, and 106, respectively;
m) SEQ ID NOs: 87, 76, 97, 107, 89, and 90, respectively;
n) SEQ ID NOs: 87, 76, 98, 104, 89, and 90, respectively;
o) SEQ ID NOs: 87, 76, 98, 88, 89, and 105, respectively;
p) SEQ ID NOs: 87, 76, 98, 88, 89, and 106, respectively;
q) SEQ ID NOs: 87, 76, 98, 107, 89, and 90, respectively;
r) SEQ ID NOs: 87, 76, 101, 104, 89, and 90, respectively;
s) SEQ ID NOs: 87, 76, 101, 88, 89, and 105, respectively;
t) SEQ ID NOs: 87, 76, 101, 88, 89, and 106, respectively; or
u) SEQ ID NOs: 87, 76, 101, 107, 89, and 90, respectively.

6. The anti-TNFα antibody or antigen-binding portion of claim 5, wherein said antibody comprises a heavy chain variable domain (VH) and a light chain variable domain (VL), wherein said VH and VL comprise:
a) SEQ ID NOs: 30 and 16, respectively;
b) SEQ ID NOs: 32 and 16, respectively;
c) SEQ ID NOs: 34 and 16, respectively;
d) SEQ ID NOs: 36 and 38, respectively;
e) SEQ ID NOs: 14 and 40, respectively;
f) SEQ ID NOs: 14 and 42, respectively;
g) SEQ ID NOs: 14 and 44, respectively;
h) SEQ ID NOs: 14 and 46, respectively;
i) SEQ ID NOs: 14 and 48, respectively;
j) SEQ ID NOs: 30 and 40, respectively;
k) SEQ ID NOs: 30 and 42, respectively;
l) SEQ ID NOs: 30 and 44, respectively;
m) SEQ ID NOs: 30 and 46, respectively;
n) SEQ ID NOs: 32 and 40, respectively;
o) SEQ ID NOs: 32 and 42, respectively;
p) SEQ ID NOs: 32 and 44, respectively;
q) SEQ ID NOs: 32 and 46, respectively;
r) SEQ ID NOs: 34 and 40, respectively;
s) SEQ ID NOs: 34 and 42, respectively;
t) SEQ ID NOs: 34 and 44, respectively; or
u) SEQ ID NOs: 34 and 46, respectively.

7. An anti-TNFα antibody or antigen-binding portion thereof that comprises heavy chain (HC) CDR1-3 and light chain (LC) CDR1-3 comprising:
a) SEQ ID NOs: 81, 76, 109, 83, 79, and 80, respectively;
b) SEQ ID NOs: 81, 76, 110, 83, 79, and 80, respectively;
c) SEQ ID NOs: 81, 76, 111, 83, 79, and 80, respectively;
d) SEQ ID NOs: 81, 76, 82, 112, 79, and 80, respectively;
e) SEQ ID NOs: 81, 76, 82, 83, 79, and 99, respectively;
f) SEQ ID NOs: 81, 76, 82, 83, 79, and 113, respectively;
g) SEQ ID NOs: 81, 76, 82, 83, 79, and 100, respectively;
h) SEQ ID NOs: 81, 76, 82, 114, 79, and 80, respectively;
i) SEQ ID NOs: 81, 76, 82, 83, 79, and 115, respectively;
j) SEQ ID NOs: 81, 76, 82, 112, 79, and 116, respectively;
k) SEQ ID NOs: 81, 76, 82, 117, 79, and 80, respectively;
l) SEQ ID NOs: 81, 76, 82, 118, 79, and 80, respectively; or
m) SEQ ID NOs: 81, 76, 82, 119, 79, and 80, respectively.

8. The anti-TNFα antibody or antigen-binding portion of claim 7, wherein said antibody comprises a heavy chain variable domain (VH) and a light chain variable domain (VL), wherein said VH and VL comprise:
a) SEQ ID NOs: 50 and 8, respectively;
b) SEQ ID NOs: 52 and 8, respectively;
c) SEQ ID NOs: 54 and 8, respectively;
d) SEQ ID NOs: 6 and 56, respectively;
e) SEQ ID NOs: 6 and 58, respectively;
f) SEQ ID NOs: 6 and 60, respectively;
g) SEQ ID NOs: 6 and 62, respectively;
h) SEQ ID NOs: 6 and 64, respectively;
i) SEQ ID NOs: 6 and 66, respectively;
j) SEQ ID NOs: 6 and 68, respectively;
k) SEQ ID NOs: 6 and 70, respectively;
l) SEQ ID NOs: 6 and 72, respectively; or
m) SEQ ID NOs: 6 and 74, respectively.

9. The anti-TNFα antibody or antigen-binding portion of any one of claims 5-8, wherein said antibody or antigen-binding portion is monovalent.

10. The anti-TNFα antibody of claim 6 or 8, wherein said antibody is monovalent and comprises
a) a monovalent antigen-binding protein that comprises an HC comprising said VH and an LC comprising said VL; and
b) a truncated HC lacking the variable domain and CH1 domain;
wherein the antigen-binding protein HC and the truncated HC are capable of dimerization.

11. A monovalent anti-TNFα antibody comprising
a) a monovalent antigen-binding protein that comprises an HC comprising a VH with the amino acid sequence of SEQ ID NO: 50 and an LC comprising a VL with the amino acid sequence of SEQ ID NO: 8; and
b) a truncated HC lacking the variable domain and CH1 domain;
wherein the antigen-binding protein HC and the truncated HC are capable of dimerization.

12. A monovalent anti-TNFα antibody comprising
a) a monovalent antigen-binding protein that comprises an HC comprising a VH with the amino acid sequence of SEQ ID NO: 54 and an LC comprising a VL with the amino acid sequence of SEQ ID NO: 8; and
b) a truncated HC lacking the variable domain and CH1 domain;
wherein the antigen-binding protein HC and the truncated HC are capable of dimerization.

13. A monovalent anti-TNFα antibody comprising
a) a monovalent antigen-binding protein that comprises an HC comprising a VH with the amino acid sequence of SEQ ID NO: 6 and an LC comprising a VL with the amino acid sequence of SEQ ID NO: 56; and
b) a truncated HC lacking the variable domain and CH1 domain;
wherein the antigen-binding protein HC and the truncated HC are capable of dimerization.

14. A monovalent anti-TNFα antibody comprising
a) a monovalent antigen-binding protein that comprises an HC comprising a VH with the amino acid sequence of SEQ ID NO: 6 and an LC comprising a VL with the amino acid sequence of SEQ ID NO: 64; and
b) a truncated HC lacking the variable domain and CH1 domain;
wherein the antigen-binding protein HC and the truncated HC are capable of dimerization.

15. A monovalent anti-TNFα antibody comprising
a) a monovalent antigen-binding protein that comprises an HC comprising a VH with the amino acid sequence of SEQ ID NO: 6 and an LC comprising a VL with the amino acid sequence of SEQ ID NO: 68; and
b) a truncated HC lacking the variable domain and CH1 domain;
wherein the antigen-binding protein HC and the truncated HC are capable of dimerization.

16. A monovalent anti-TNFα antibody comprising
a) a monovalent antigen-binding protein that comprises an HC comprising a VH with the amino acid sequence of SEQ ID NO: 14 and an LC comprising a VL with the amino acid sequence of SEQ ID NO: 44; and
b) a truncated HC lacking the variable domain and CH1 domain;
wherein the antigen-binding protein HC and the truncated HC are capable of dimerization.

17. The monovalent anti-TNFα antibody of any one of claims 10-16, wherein the antigen-binding protein HC and the truncated HC comprise knobs-into-holes modifications, optionally wherein the antigen-binding protein HC is of isotype subclass IgG1 and comprises mutations T366S, L368A, and Y407A in the CH3 domain and the truncated HC is of isotype subclass IgG1 and comprises the mutation T366W in the CH3 domain, wherein the residues are numbered according to the Eu system.

18. The monovalent anti-TNFα antibody of any one of claims 10-17, wherein the antigen-binding protein HC is of isotype subclass IgG1 and comprises the mutation Y349C and the truncated HC is of isotype subclass IgG1 and comprises the mutation S354C, wherein the residues are numbered according to the Eu system.

19. The anti-TNFα antibody of claim 6 or 8, wherein said antibody is monovalent and comprises
a) a single-chain variable fragment (scFv) that comprises said VH and said VL, linked to an Fc monomer; and
b) a truncated HC lacking the variable domain and CH1 domain;
wherein the Fc monomer linked to the scFv, and the truncated HC, are capable of dimerization.

20. The monovalent anti-TNF antibody of claim 19, wherein the Fc monomer linked to the scFv, and the truncated HC, comprise knobs-into-holes modifications, optionally wherein the Fc monomer linked to the scFv is of isotype subclass IgG1 and comprises mutations T366S, L368A, and Y407A in the CH3 domain, and the truncated HC is of isotype subclass IgG1 and comprises the mutation T366W in the CH3 domain, wherein the residues are numbered according to the Eu system.

21. The monovalent anti-TNFα antibody of claim 19 or 20, wherein the Fc monomer linked to the scFv is of isotype subclass IgG1 and comprises the mutation Y349C, and the truncated HC is of isotype subclass IgG1 and comprises the mutation S354C, wherein the residues are numbered according to the Eu system.

22. The anti-TNFα antibody or antigen-binding portion of any one of claims 5-21, wherein said antibody or antigen-binding portion has a binding affinity for human TNFα that is lower at pH 6.0 than at pH 7.4.

23. The anti-TNFα antibody or antigen-binding portion of any one of claims 1-22, wherein said antibody or antigen-binding portion
a) undergoes less degradation in vivo;
b) undergoes increased recycling to the cell surface in vivo;
c) has a longer half-life in vivo;
d) is less immunogenic in vivo; or
e) any combination of a)-d);
than an antibody comprising VH and VL amino acid sequences of SEQ ID NOs: 2 and 4, respectively; SEQ ID NOs: 6 and 8, respectively; SEQ ID NOs: 10 and 12, respectively; or SEQ ID NOs: 14 and 16, respectively; optionally wherein said antibody or antigen-binding portion does not form large immune complexes.

24. A bispecific binding molecule having the binding specificity of an anti-TNFα antibody of any one of claims 1-23 and the binding specificity of a second, distinct antibody.

25. The bispecific binding molecule of claim 24, wherein the second antibody is an anti-IL17A antibody, an anti-IL23 antibody, or an anti-angiopoietin 2 (Ang2) antibody.

26. An immunoconjugate comprising an anti-TNFα antibody or antigen-binding portion of any one of claims 1-23 linked to a therapeutic agent.

27. The immunoconjugate of claim 26, wherein the therapeutic agent is an anti-inflammatory or immunosuppressive agent, optionally wherein the therapeutic agent is a steroid.

28. Isolated nucleic acid molecule(s) comprising nucleotide sequences that encode the heavy and light chain sequences of the anti-TNFα antibody or antigen-binding portion of any one of claims 1-23.

29. The isolated nucleic acid molecule(s) of claim 28, comprising the nucleotide sequences of:
a) SEQ ID NOs: 29 and 15;
b) SEQ ID NOs: 31 and 15;
c) SEQ ID NOs: 33 and 15;
d) SEQ ID NOs: 35 and 37;
e) SEQ ID NOs: 13 and 39;
f) SEQ ID NOs: 13 and 41;
g) SEQ ID NOs: 13 and 43;
h) SEQ ID NOs: 13 and 45;
i) SEQ ID NOs: 13 and 47;
j) SEQ ID NOs: 29 and 39;
k) SEQ ID NOs: 29 and 41;
l) SEQ ID NOs: 29 and 43;
m) SEQ ID NOs: 29 and 45;
n) SEQ ID NOs: 31 and 39;
o) SEQ ID NOs: 31 and 41;
p) SEQ ID NOs: 31 and 43;
q) SEQ ID NOs: 31 and 45;
r) SEQ ID NOs: 33 and 39;
s) SEQ ID NOs: 33 and 41;
t) SEQ ID NOs: 33 and 43;
u) SEQ ID NOs: 33 and 45;
v) SEQ ID NOs: 49 and 7;
w) SEQ ID NOs: 51 and 7;
x) SEQ ID NOs: 53 and 7;
y) SEQ ID NOs: 5 and 55;
z) SEQ ID NOs: 5 and 57;
aa) SEQ ID NOs: 5 and 59;
bb) SEQ ID NOs: 5 and 61;
cc) SEQ ID NOs: 5 and 63;
dd) SEQ ID NOs: 5 and 65;
ee) SEQ ID NOs: 5 and 67;
ff) SEQ ID NOs: 5 and 69;
gg) SEQ ID NOs: 5 and 71; or
hh) SEQ ID NOs: 5 and 73.

30. Vector(s) comprising the isolated nucleic acid molecule(s) of claim 28 or 29, wherein the vector(s) further comprise expression control sequence(s) linked operatively to the isolated nucleic acid molecule(s).

31. A host cell comprising a nucleotide sequence that encodes the heavy chain sequence(s), and a nucleotide sequence that encodes the light chain sequence, of the anti-TNFα antibody or antigen-binding portion of any one of claims 1-23.

32. The host cell of claim 31, wherein said host cell comprises the isolated nucleic acid molecule(s) of claim 29.

33. A method for producing an anti-TNFα antibody or an antigen-binding portion thereof, comprising providing the host cell of claim 31 or 32, culturing said host cell under conditions suitable for expression of the antibody or antigen-binding portion, and isolating the resulting antibody or antigen-binding portion.

34. A pharmaceutical composition comprising the anti-TNFα antibody or antigen-binding portion of any one of claims 1-23, the bispecific binding molecule of claim 24 or 25, or the immunoconjugate of claim 26 or 27, and a pharmaceutically acceptable excipient.

35. A method for treating an autoimmune or inflammatory condition in a patient in need thereof, comprising administering to said patient a therapeutically effective amount of the anti-TNFα antibody or antigen-binding portion of any one of claims 1-23, the bispecific binding molecule of claim 24 or 25, or the immunoconjugate of claim 26 or 27.

36. Use of the anti-TNFα antibody or antigen-binding portion of any one of claims 1-23, the bispecific binding molecule of claim 24 or 25, or the immunoconjugate of claim 26 or 27, for the manufacture of a medicament for treating an autoimmune or inflammatory condition in a patient in need thereof.

37. The anti-TNFα antibody or antigen-binding portion of any one of claims 1-23, the bispecific binding molecule of claim 24 or 25, or the immunoconjugate of claim 26 or 27, for use in treating an autoimmune or inflammatory condition in a patient in need thereof.

38. The method; use; or anti-TNFα antibody or antigen-binding portion, bispecific binding molecule, or immunoconjugate for use; of any one of claims 35-37, wherein the autoimmune or inflammatory condition is rheumatoid arthritis, psoriatic arthritis, plaque psoriasis, ankylosing spondylitis, axial spondyloarthritis, Crohn's disease, ulcerative colitis, hidradenitis suppurativa, polyarticular juvenile idiopathic arthritis, panuveitis, or Alzheimer's disease.

39. The method; use; or anti-TNFα antibody or antigen-binding portion, bispecific binding molecule, or immunoconjugate for use; of any one of claims 35-38, wherein the patient is treated with an additional therapeutic agent.

40. The method; use; or anti-TNFα antibody or antigen-binding portion, bispecific binding molecule, or immunoconjugate for use; of claim 39, wherein the additional therapeutic agent is an anti-inflammatory or immunosuppressive agent, optionally wherein the additional therapeutic agent is methotrexate.

41. A kit comprising the anti-TNFα antibody or antigen-binding portion of any one of claims 1-23, the bispecific binding molecule of claim 24 or 25, or the immunoconjugate of claim 26 or 27.

42. The kit of claim 41, for use in a treatment in accordance with the method of any one of claims 35 and 38-40.

43. An article of manufacture comprising the anti-TNFα antibody or antigen-binding portion of any one of claims 1-23, the bispecific binding molecule of claim 24 or 25, or the immunoconjugate of claim 26 or 27, wherein said article of manufacture is suitable for treating an autoimmune or inflammatory condition in a patient in need thereof.

44. The article of manufacture of claim 43, wherein the treatment is in accordance with the method of any one of claims 35 and 38-40.
